(19) United States
(12) Patent Application Publication
(10) Pub. No.: US 2012/0076762 A1
(43) Pub. Date: Mar. 29, 2012

(54) INDUCED PLURIPOTENT STEM CELL GENERATION USING TWO FACTORS AND P53 INACTIVATION

(75) Inventors: Teruhisa Kawamura, Kyoto (JP); Jotaro Suzuki, Ibaraki (JP); Yunyuan V. Wang, San Diego, CA (US); Angel Raya, Barcelona (ES); Geoffrey M. Wahl, San Diego, CA (US); Juan Carlos Izpisua Belmonte, La Jolla, CA (US)

(73) Assignee: THE SALK INSTITUTE FOR BIOLOGICAL STUDIES, La Jolla, CA (US)

(21) Appl. No.: 13/259,644

(22) PCT Filed: Mar. 24, 2010
(86) PCT No.: PCT/US10/28541

§ 371 Date: Dec. 19, 2011

Related U.S. Application Data
(60) Provisional application No. 61/163,386, filed on Mar. 25, 2009.

(51) Int. Cl. A61K 35/12; C12N 5/10; A61P 43/00; C12N 15/85

(52) U.S. Cl. 424/93.21; 435/455; 435/366

(57) ABSTRACT

Methods and compositions are provided for, inter alia, the generation of induced pluripotent stem cells. Induced pluripotent stem cells may be generated by reprogramming and inhibition of p53. Further, useful intermediates for the generation of induced pluripotent stem cells are also provided.

### CROSS-REFERENCES TO RELATED APPLICATIONS

This application claims the benefit of U.S. Provisional Application No. 61/163,386, filed Mar. 25, 2009, which is incorporated herein by reference in its entirety and for all purposes.

### STATEMENT AS TO RIGHTS TO INVENTIONS MADE UNDER FEDERALLY SPONSORED RESEARCH AND DEVELOPMENT

The invention was made with government support under RO1CA061449 awarded by the National Institutes of Health. The Government has certain rights in the invention.

### BACKGROUND OF THE INVENTION

Reprogramming somatic cells to induced pluripotent stem (iPS) cells has been accomplished through expression of a combination of pluripotency factors and oncogenes, but the low frequency and tendency to induce malignant transformation compromise the utility of this powerful approach for patient use. Previous work showed that mouse and human cells can be directly converted into pluripotent cells, [induced-pluripotent stem cells (iPS cells)], using forced over-expression of three (Oct4/Sox2/Klf4) or four (Oct4/Sox2/c-Myc/Klf4 or Oct4/Sox2/Lin28/Nanog) factors¹⁻⁸. However, in most protocols, the two oncogenes c-Myc and Klf4 are employed, which induce cellular transformation and cancer upon generation of chimeric animals⁹. The p53 pathway acts as a barrier to cancer through induction of apoptosis or cell cycle arrest in response to a variety of stress signals, including over-expressed oncogenes such as c-Myc. Klf4 can either activate or antagonize p53, depending on the cell type used and expression level¹⁰. Further, prior results have shown that germ cells can be spontaneously reprogrammed in the absence of p53¹¹. Consequently, reprogramming efficiency is likely reduced through oncogene-mediated activation of the p53 pathway. The methods and compositions described herein overcome these and other problems in the art.

### BRIEF SUMMARY OF THE INVENTION

The present invention provides, inter alia, highly efficient methods and compositions for making and using an induced pluripotent stem cell. The pluripotent stem cell may be generated by transfection of a non-pluripotent cell with nucleic acids encoding an Oct4 protein and a Sox2 protein, and by inhibiting p53 expression and/or function of the non-pluripotent cell.

In one aspect, a method for preparing a method for preparing an induced pluripotent stem cell is provided. The method includes transfecting a non-pluripotent cell with a nucleic acid encoding an Oct4 protein, a nucleic acid encoding a Sox2 protein and a nucleic acid encoding a p53 inhibitor to form a transfected non-pluripotent cell. The transfected non-pluripotent cell is allowed to divide and thereby forms the induced pluripotent stem cell.

In another aspect, a method for preparing an induced pluripotent stem cell is provided. The method includes transfecting a p53-deficient non-pluripotent cell with a nucleic acid encoding an Oct4 protein and a nucleic acid encoding a Sox2 protein to form a transfected p53-deficient non-pluripotent cell. The transfected p53-deficient non-pluripotent cell is allowed to divide and thereby forms the induced pluripotent stem cell.

In another aspect, a method for preparing an induced pluripotent stem cell is provided. The method includes introducing a p53 inhibitor to a non-pluripotent cell. The non-pluripotent cell is transfected with a nucleic acid encoding an Oct4 protein and a nucleic acid encoding a Sox2 protein to form a transfected non-pluripotent cell. The transfection of the non-pluripotent cell is performed before, after or at the same time of introducing the p53 inhibitor to the non-pluripotent cell. The transfected non-pluripotent cell is allowed to divide and thereby forms the induced pluripotent stem cell.

In one aspect, an induced pluripotent stem cell is prepared according to the methods provided herein.

In another aspect, a non-pluripotent cell including a nucleic acid encoding an Oct4 protein, a nucleic acid encoding a Sox2 protein and a nucleic acid encoding a p53 inhibitor is provided.

In another aspect, a p53-deficient non-pluripotent cell including a nucleic acid encoding an Oct4 protein and a nucleic acid encoding a Sox2 protein is provided.

In another aspect, a non-pluripotent cell including a nucleic acid encoding an Oct4 protein, a nucleic acid encoding a Sox2 protein and a p53 inhibitor is provided.

In one aspect, a method of treating a mammal in need of tissue repair is provided. The method includes administering an induced pluripotent stem cell to the mammal. The induced pluripotent stem cell is allowed to divide and differentiate into somatic cells in the mammal thereby providing tissue repair in the mammal. The induced pluripotent stem cell may be prepared by a process that includes transfecting a non-pluripotent cell with a nucleic acid encoding an Oct4 protein, a nucleic acid encoding a Sox2 protein and a nucleic acid encoding a p53 inhibitor to form a transfected non-pluripotent cell. The transfected non-pluripotent cell is allowed to divide and thereby forms the induced pluripotent stem cell. The induced pluripotent stem cell may be prepared by a process that includes transfecting a p53-deficient non-pluripotent cell with a nucleic acid encoding an Oct4 protein and a nucleic acid encoding a Sox2 protein to form a transfected p53-deficient non-pluripotent cell. The transfected p53-deficient non-pluripotent cell is allowed to divide and thereby forms the induced pluripotent stem cell. The induced pluripotent stem cell may be prepared by a process that includes introducing a p53 inhibitor to a non-pluripotent cell. The non-pluripotent cell is transfected with a nucleic acid encoding an Oct4 protein and a nucleic acid encoding a Sox2 protein to form a transfected non-pluripotent cell. The transfection of the non-pluripotent cell is performed before, after or at the same time of introducing the p53 inhibitor to the non-pluripotent cell. The transfected non-pluripotent cell is allowed to divide and thereby forms the induced pluripotent stem cell.

In another aspect, a method for producing a somatic cell is provided. The method includes contacting an induced pluripotent stem cell with a cellular growth factor. The induced pluripotent stem cell is allowed to divide, thereby forming the somatic cell. The induced pluripotent stem cell may be prepared by a process that includes transfecting a non-pluripotent cell with a nucleic acid encoding an Oct4 protein, a nucleic acid encoding a Sox2 protein and a nucleic acid encoding a p53 inhibitor to form a transfected non-pluripotent cell. The transfected non-pluripotent cell is allowed to divide and thereby forms the induced pluripotent stem cell. The induced pluripotent stem cell may be prepared by a process that includes transfecting a p53-deficient non-pluripotent cell with a nucleic acid encoding an Oct4 protein and a nucleic acid encoding a Sox2 protein to form a transfected p53-deficient non-pluripotent cell. The transfected p53-deficient non-pluripotent cell is allowed to divide and thereby forms the induced pluripotent stem cell. The induced pluripotent stem cell may be prepared by a process that includes introducing a p53 inhibitor to a non-pluripotent cell. The non-pluripotent cell is transfected with a nucleic acid encoding an Oct4 protein and a nucleic acid encoding a Sox2 protein to form a transfected non-pluripotent cell. The transfection of the non-pluripotent cell is performed before, after or at the same time of introducing the p53 inhibitor to the non-pluripotent cell. The transfected non-pluripotent cell is allowed to divide and thereby forms the induced pluripotent stem cell.

### BRIEF DESCRIPTION OF THE DRAWINGS

FIG. 1: Increased generation of iPS cells by blocking p53 and p21. (FIG. 1a) Mouse embryonic fibroblasts (MEFs) were infected by retroviruses encoding 3 transcription factors (Oct4/Sox2/Klf4), 2 factors (Oct4/Sox2), Klf4, c-Myc or GFP. Four days after infection, the protein levels of p53, Arf, and p21 were analyzed by Western Blotting. α-tubulin was used as a loading control. (FIG. 1b) Mouse embryonic fibroblasts (MEFs) were infected by retroviruses encoding three transcription factors (Oct4/Sox2/Klf4) in combination with mock, control shRNA (GFP) and p53 shRNA (#1 and #2). Emerging colonies of iPS cells were visualized by immunostaining with anti-Nanog antibody using an Avidin Biotin Complex (ABC) method. (FIG. 1c) The fold change in the number of Nanog-positive colonies compared to mock (n=4). For all figures in this study, error bars indicate s.d. (FIG. 1d) Effect of Nutlin 3a on the number of Nanog-positive colonies by 3 factors in combination with mock, p53, and p21 shRNA. Cells were treated by Nutlin 3a (0, 3, 10 μM) starting from day 4. Protein levels of p53 and α-Tubulin (loading control) after treatment with Nutlin 3a for 3 hours is shown. (FIG. 1e) Immunostaining of Nanog positive colonies generated from p53(+/+), p53(+/−) and p53(−/−) MEFs by three factors showed p53 dose-dependent decrease of colony number. (FIG. 1f) Retroviral infection of p53 into p53(−/−) MEF decreased the number of Nanog-positive colonies induced by three factors. Protein levels of p53, p21 and α-Tubulin (loading control) on day 3 after infection are shown. (FIG. 1g) Nutlin 3a dramatically reduced reprogramming of p53(+/+) MEFs, but not on p53(−/−) MEF. (FIG. 1h) Fold change in the number of Nanog-positive colonies by p21 shRNA (n=4). Protein levels of p21 and α-Tubulin (loading control) on day 4 after infection are shown.

FIG. 2: Modulation of p53 activity alters reprogramming efficiency. (FIG. 2a) Fold change in the number of 3-F induced Nanog-positive colonies by Arf shRNA compared to control shRNA (n=3). Protein levels of Arf, Ink4a and α-Tubulin (loading control) on day 4 after infection are shown. (FIG. 2b) Fold change in the number of 3-F induced Nanog-positive colonies by Arf/Ink4a shRNA compared to control shRNA (n=3). Protein levels of Arf, Ink4a and α-Tubulin (loading control) on day 4 after infection are shown. (FIG. 2c) MEFs isolated from wild type (+/+), Mdmx-3SA heterozygous (3SA/+) and homozygous (3SA/3SA) mice were exposed to 5Gy of gamma-irradiation for 4 hours. p21 mRNA levels were analyzed by QPCR. Results were normalized to untreated WT samples for fold induction. (FIG. 2d) 3-F induced Nanog-positive colonies in wild type (+/+) and homozygous (3SA/3SA) mice (n=3).

FIG. 3: Generation of 2F-p53KD-iPS cells by p53 downregulation. (FIG. 3a) Morphology and GFP fluorescence of PO colonies of 2F-p53KD-iPS cells. Clone #2 and #6 had weaker GFP fluorescence when compared to the other clones. (FIG. 3b) Morphology and GFP fluorescence of 2F-p53KD-iPS cell lines. GFP expression is silenced in clone #6. (FIG. 3c) Alkaline phosphatase staining (FIG. 3d) Nanog-immunostaining of 2F-p53KD-iPS cell lines. DAPI was used to visualize cell nuclei. (FIG. 3e) Protein levels of Nanog, Oct4, Sox2, Klf4, c-Myc, p53 in 2F-p53KD-iPS cell lines are shown. α-Tubulin was used as loading control. (FIG. 3f) Methylation analysis of Oct4 and Nanog promoters in 2F-p53KD-iPS cells, MEFs and ES cells. Black circles indicate methylated CpGs and white circles indicate unmethylated CpGs. (FIG. 3g) Quantitative PCR analysis of ES cell marker genes. To investigate silencing of viral transgenes (Oct4 and Sox2), primer sets that specifically amplify endogenous Oct4 or Sox2 were used. Values were standardized to GAPDH, and normalized to ES cells.

FIG. 4: In vitro and in vivo differentiation of 2F-p53 KD iPS colonies. (FIG. 4a) Embryoid bodies (EBs) of 2F-p53KD-iPS cell clones on day 6 of differentiation. (FIG. 4b) EBs were transferred to gelatinized dishes on day 3 to 5 for further differentiation. On day 14, EBs were subjected to immunofluorescence for α-fetoprotein (AFP)/Foxa2 (endoderm), α-sarcomeric actin/GATA4 (mesoderm) and Tuj1/GFAP (ectoderm). (FIG. 4c) QPCR analysis for differentiation marker genes of the three germ layers (AFP, HNF1 and GATA6 for endoderm, GATA4, alpha cardiac actin, and smooth muscle (SM) actin for mesoderm, Cdx2 and Mfap2 for ectoderm) in 2F-p53KD-iPS cells on d14 of differentiation. (FIG. 4d) Immunofluorescence of teratoma from 2F-p53KD-iPS cells by antibodies against AFP/Foxa2 (endoderm), α-sarcomeric actinin/Chondroitin (mesoderm), Tuj1/GFAP (ectoderm) showed spontaneous differentiation into all three germ layers. (FIG. 4e) Adult chimeric mice obtained from 2F-p53 KD iPS lines (#1 and #6) and non-chimeric mouse in C57BL/6J host blastocysts. Brown hair is originated from iPS cells.

FIG. 5: Downregulation of p53 activity increases reprogramming efficiency of human somatic cells. (FIG. 5a) Human embryonic fibroblasts were infected with retroviruses encoding Oct4/Sox2/Klf4 (3-F) or Oct4/Sox2/Klf4/c-Myc (4-F) factors in combination with lentiviruses expressing control- or p53-shRNA. Emerging colonies of iPS cells were visualized by immunostaining with anti-Nanog antibody using an ABC method. Lentiviruses encoding p53 shRNA efficiently knocked down p53 expression, as judged by Western blot analysis (lower panels). α-Tubulin was used as a loading control. (FIG. 5b) Human primary keratinocytes were co-infected with 4-F and retroviruses expressing GFP or p53-DD. 5×10⁴ infected cells were plated on 10-cm dishes and stained for AP activity after 2 weeks. Expression of p53-DD resulted in stabilization of wild-type p53, as visualized by Western blot analysis (lower panels). Actin was used as a loading control. (FIG. 5c) The bars represent the average number of iPS-like colonies obtained from 10⁴ keratinocytes reprogrammed with 3-F or 4-F and retroviruses encoding GFP or p53-DD, in the absence or presence of the p53 agonist Nutlin3a (n=3). iPS-like colonies were scored as having hES-like morphology and positive AP staining. Due to the numerous colonies generated in 4-F p53-DD keratinocytes, quantification was done using 10⁴ cells. (FIGS. 5d-e) Colonies of human keratinocyte-derived iPS cells generated by 3-F and p53-DD display strong immunoreactivity for pluripotency-associated transcription factors and surface markers (FIG. 5d) and differentiate in vitro into cell types that express markers of endoderm (α-fetoprotein, FoxA2), mesoderm (GATA4, sarcomeric α-actinin), and ectoderm (Tuj1, TH) (FIG. 5e).

FIG. 6: γ-H2AX focus formation after Yamanaka factors expression. MEFs were infected with 4F(Oct4/Sox2/Klf4/c-Myc), 3F(Oct4/Sox2/Klf4), 2F(Oct4/Sox2), GFP or c-Myc retroviruses. After 4 or 6 days, cells were fixed and stained by anti-phospho-Histone H2A.X (Ser139) antibody in combination with anti-rabbit antibody conjugated with TRITC. Nuclei were visualized by DAPI. The number of cells containing >10 γ-H2AX focuses in nuclei were counted under the microscope. Data was shown as % of cells including >10 γ-H2AX focus in nuclei versus total cells (n=3).

FIG. 7: The level of p53 and p21 protein in human and mouse cells previously used for reprogramming. Equal amount of proteins (30 mg) were subjected to SDS-PAGE and each protein level was analyzed by western blotting using each antibody. Mouse embryonic fibroblasts were analyzed at passage 3 of derivation. Mouse hepatocytes were isolated by two-steps collagenase perfusion and cultured in the presence of EGF. Mouse neural stem cells are neurospheres from embryonic forebrain (E12.5). Human foreskin fibroblasts and IMR90 were analyzed at passage 6 of derivation. Human keratinocytes were isolated as previously described (Aasen, T. et al. (2008). Nat. Biotechnol. 26, 1276-84). Human neural progenitors were differentiated from human ES cells (HUES6). Human ES cells are HUES9.

FIG. 8: Human p53/p21 level after 3-F infection with p53 shRNA in keratinocyte and fibroblast. Human keratinocytes or foreskin fibroblasts were infected (day 0) with retroviruses for 3-F (Oct4, Sox-2 and KLF4) and/or lentiviruses encoding a p53 shRNA or and empty vector control as indicated. For the non-infected control, cells were grown in exactly the same conditions. Protein samples were collected at day five; the protein concentration was carefully standardized and the samples were analyzed by western-blot as indicated in the materials and methods section. Membranes were stained using antibodies for human p21 and tubulin as a loading control.

FIG. 9: Highly efficient lentivirus-mediated shRNA expression in MEFs. pLVTHM-short hairpin RNA (shRNA) expressing lentivirus vector harbors GFP as a reporter under elongation factor promoter. After infection of this lentivirus together with retrovirus of three factors (Oct4/Sox2/Klf4), the majority of cells became positive for GFP. Cells were infected by retrovirus and lentivirus in the following proportion: pMX-Oct4:pMX-Sox2:pMX-Klf4:pLVTHM-shRNA=1:1:1:4.

FIG. 10: RT-PCR and QPCR analysis of three transgenes (Oct4/Sox2/Klf4), p53, and p21 four days after infection. (FIG. 10a) mRNA expression of three transgenes in MEFs infected with 3 factors (Oct4, Sox2, and Klf4) by retrovirus was analyzed by RT-PCR (n=3 or 4). The mRNA levels of the three factors were similar in all samples. GAPDH was used as an experimental control. (FIG. 10b) Western blotting shows that the protein levels of the three transgenes were similar among all the groups. (FIG. 10c) Decrease in p53 and p21 mRNA levels by each shRNA was shown by QPCR analysis. Data was normalized by GAPDH levels.

FIG. 11: Senescence-associated b-galactosidase. Senescence-associated b-gal activity was histochemically detected as previously described (Dimri G P et al. (1995). Proc Natl Acad Sci USA. 92, 9363-67.). p53+/+, p53−/+ and p53−/− MEFs were analyzed just before virus infection at passage 3 after derivation. MEFs were infected by mock or p53 shRNA#2 together with Oct4, Sox2 and Klf4, and senescence-associated b-gal activity was analyzed 2 days after infection. No significant difference among these cells in the intensity of senescence-associated b-gal staining. For a positive control of staining, MEFs were infected by c-Myc and cultured for 9 days after infection.

FIG. 12: No loss of heterogeneity in iPS cell lines derived from p53+/− MEF. p53+/− MEFs were infected with 3 factors (Oct4, Sox2, and Klf4) by retrovirus and established iPS cell lines. Seven independent lines of iPS cells were analyzed for p53 protein level by western blotting. For control, p53−/−, p53−/+, p53+/+MEFs and mouse ES cells were analyzed at the same time. Each lane was loaded 35 mg of total protein. α-Tubulin was utilized for loading control. All 7 independent iPS cell lines expressed p53 protein.

FIG. 13: Bcl-2 increased the efficiency of reprogramming. (FIG. 13a) MEFs were infected by retroviruses encoding 4 transcription factors (Oct4/Sox2/Klf4/c-Myc), 3 factors (Oct4/Sox2/Klf4), or 2 factors (Oct4/Sox2) with lentivirus encoding Bcl-2. Five days after infection, TUNEL-staining was performed. The percentage of apoptotic cells was analyzed (using at least five independent panels) and represented graphically. Error bars indicate s.d. (FIG. 13b) MEFs were infected by retroviruses encoding 3 factors (Oct4/Sox2/Klf4) in combination with mock or Bcl-2 expressing lentivirus. The fold change in the number of Nanog-positive colonies compared to mock (n=3) is shown. Error bars indicate s.d (n=3).

FIG. 14: Morphology and Nanog expression in mouse iPS colonies induced by ectopic expression of 3 factors (retrovirus) plus shRNA (lentivirus). Nanog expression was detected by anti-Nanog antibody with a secondary antibody conjugated with TRITC. Incorporation of lentivirus was confirmed by GFP fluorescence. Nuclei were visualized by DAPI.

FIG. 15: Characterization of mouse 3F iPS clones. First row panels show the morphology of 3F iPS clones expressing shRNAs by lentiviral infection (left: mock, middle: p53 shRNA, right: p21 shRNA). Second row panels show GFP fluorescence by shRNA-encoding lentiviruses. Third row panels show alkaline phosphatase staining Fourth row panels show Nanog immunoreactivity. Fifth row panels show nuclei staining by DAPI.

FIG. 16: Pluripotent markers and in vitro differentiation of mouse 3F iPS clones. (FIG. 16a) Analysis of pluripotent markers and (FIG. 16b) differentiation markers in established 3F iPS clones. For pluripotent markers, data from one representative clone are shown. For differentiation markers, data from 3 clones are shown. Cells were differentiated by embryoid bodies for 14 days. For ectodermal induction, EBs were treated with retinoic acid. The relative amounts of Oct4(end), Sox2(end), c-Myc, Nanog, DPPA2, DPPA5, E-ras, Zfp42, AFP, HNF1b, GATA6, GATA4, aCardiac actin, SM actin, Cdx2, Mfap2 and Nestin mRNA were quantified by QPCR. Values were standardized to GAPDH and normalized to ES cells (n=3, ±s.e.).

FIG. 17: Analysis of the cell cycle profile of 2F-p53KD-iPS cell clones. A majority of the cells in 2F-p53KD-iPS cell clones and ES cells were in S-phase (clone1=79%, clone3=77%, clone5=67%, clone6=80%, ES=76%), while only 22% of MEFs were in S phase. Four independent clones of 2F-p53KD-iPS cells, MEFs and ES cells were treated with BrdU and fixed. Cells were treated with anti-BrdU-biotin followed by avidin-APC, and incorporated BrdU was analyzed by FACS. Staining intensity for PI (x-axis) is plotted versus that for anti-BrdU-APC (y-axis). The S-phase population was calculated.

FIG. 18: Successful formation of beating embryoid bodies attached to gelatinized dishes from three independent 2F-p53KD-iPS cell clones. Pictures were taken at day 10 (Clone#1) day 14 (clone#3), and day 15 (clone#6).

FIG. 19: Genotyping of 2F-p53KD-iPS cell lines using PCR and Southern blotting. (FIG. 19a) Viral integration of Oct4, Sox2, and Klf4 were amplified from genomic DNA. Only virus-derived Oct4 and Sox2 were amplified (no viral Klf4) in 2F-p53KD-iPS cell clones, while viral Klf4 was amplified only from 3F-iPS cells. GFP was amplified from all three 2F-p53KD-iPS cell clones, indicating the integration of p53-shRNA lentivirus. GAPDH was used as an experimental control for PCR. (FIG. 19b) Genomic DNA in each clone was digested by BamHI and EcoRI and subjected to Southern blot analysis with a Klf4 cDNA probe, showing no viral integration of Klf4. Genomic DNA from 3F-iPS and MEFs (no virus infection) was used as positive and negative controls for Southern blot analysis.

FIG. 20: GeneChip expression analysis. Total RNA was isolated from MEF, 2F-p53KD-iPS cell clones (#1 and #6) and mouse ES cells using the Trizol reagent according to the manufacturer's instructions. The GeneChip microarray processing was performed by the Salk institute microarray platform according to the manufacturer's protocols (Affymetrix, Santa Clara, Calif.). The data extraction was done by the Affymetrix GCOS software v.1.4.

FIG. 21: Genotyping of chimeric mice derived from 2F-iPS cells. Genotyping of chimeric mice from 2F-p53KD-iPS cell clones (#1 and #6) and wild type (WT) mouse was performed using primers specific for viral Oct4 and Sox2 genes. GAPDH was utilized for control of PCR reaction.

FIG. 22: High-efficient lentivirus-mediated shRNA expression in HEFs. pLVTHM-short hairpin RNA (shRNA) expressing lentivirus vector harbors GFP as a reporter under elongation factor promoter. After infection of this lentivirus together with retrovirus of three or four factors (Oct4/Sox2/Klf4 with or without c-Myc), the majority of cells became positive for GFP. Cells were infected by retrovirus and lentivirus in the following proportion: pMSCV-Oct4:pMSCV-Sox2:pMSCV-Klf4(:pMSCV-cMyc):pLVTHM-shRNA=1:1:1(:1):3.

FIG. 23: Morphology and ES marker expression in human iPS cell colonies induced by ectopic expression of 4 factors (retrovirus) plus p53 shRNA (lentivirus) in HEFs. (FIGS. 23a-c), Phase-contrast images of human iPS cell colony (b) and non-iPS (granulated) cell colony (c) are shown. (FIGS. 23d-e) Nanog or TraI81 expression was detected by anti-Nanog (d) or anti-TraI81 (e) antibody with a secondary antibody conjugated with TRITC. Incorporation of lentivirus was confirmed by GFP fluorescence. Nuclei were visualized by DAPI.

FIG. 24: Characterization of human iPS cell lines derived from HEFs. First row panels show the morphology of 3-F or 4-F human iPS cell clones expressing p53 shRNAs by lentiviral infection. Second row panels show GFP fluorescence by shRNA-encoding lentiviruses. Third row panels show Nanog-immunoreactivity merged with nuclei staining by DAPI.

FIG. 25: Embryoid body formation of human iPS cell derived from HEFs. Phase contrast images and GFP-fluorescence of Embryoid bodies from established human iPS clones (2 representative lines of Human 3F-p53KD-iPS cell and 2 representative lines of Human 4F-p53KD-iPS cell) are shown.

FIG. 26: Pluripotent markers and in vitro differentiation of human iPS cell lines derived from HEFs. (FIG. 26a) PCR Analysis of pluripotent markers in established human iPS cell lines. Representative human iPS cell lines are 3F#1 established by 3-F infection and 4F#8, #4, #13 established by 4-F infection. For pluripotent markers, Oct4, Sox2, Nanog, DPPA2, DPPA4, Zafp42, GDF3, TDGF were analyzed. For the control of PCR reaction, GAPDH was utilized. (FIG. 26b) For differentiation markers, data from clone 4F#4 is shown. Cells were differentiated by embryoid bodies for 8-10 days. The relative amounts of PAX6, MAP2, Cdx2 (ectoderm), Msx1 (mesoderm), GATA6 and AFP (endoderm) mRNA were quantified by QPCR. Values were standardized to GAPDH and normalized to undifferentiated cells.

FIG. 27: FIGS. 27a-b depict embryoid body formation and characterization of differentiation marker expressions in 2F-p53KD-iPS cell lines. (FIG. 27a) Embryoid bodies (EBs) of 2F-p53KD-iPS cell clones on day 6 of differentiation. (FIG. 27b) QPCR analysis for differentiation marker genes of the three germ layers (AFP, HNF1 and GATA6 for endoderm, GATA4, alpha cardiac actin, and smooth muscle (SM) actin for mesoderm, Cdx2 and Mfap2 for ectoderm) in 2F-p53KD-iPS cells on d14 of differentiation.

FIG. 28: FIGS. 28a-d depict morphology and ES marker expression in human iPS cell colonies induced by ectopic expression of 2 factors (retrovirus Oct4 and Sox2) plus p53 shRNA (lentivirus) in HEFs. (FIG. 28a) Nanog-positive colony after virus transduction. (FIG. 28b) Phase-contrast images of cloned human 2F-iPS cells. (FIGS. 28c-d): Nanog or TraI81 expression was detected by anti-Nanog (FIG. 28c) or anti-TraI81 (FIG. 28d) antibody with a secondary antibody conjugated with TRITC. Nuclei were visualized by DAPI.

FIG. 29: FIG. 29 depicts the p53 pathway limitation on reprogramming efficiency. Reprogramming factors (3-F or 4-F) produce a “reprogramming stress” in somatic cells that activates the p53 pathway. This stress is caused by proto-oncogene over-expression. While the exact causes of p53 activation remain to be determined, chromatin remodeling and DNA damage are likely contributors. p53 pathway activation reduces reprogramming efficiency by activating cell cycle arrest and apoptotic responses to prevent cell division. Consequently, eliminating p53 significantly increases reprogramming. Transient p53 inhibition could facilitate development of therapeutically beneficial reprogramming applications.

### DETAILED DESCRIPTION OF THE INVENTION

### I. Definitions

Unless defined otherwise, technical and scientific terms used herein have the same meaning as commonly understood by a person of ordinary skill in the art. See, e.g., Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST/ or the like. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but to not other sequences. Stringent conditions are sequence-dependent and will be different in different circumstances. Longer sequences hybridize specifically at higher temperatures. An extensive guide to the hybridization of nucleic acids is found in Tijssen, TECHNIQUES IN BIOCHEMISTRY AND MOLECULAR BIOLOGY—HYBRIDIZATION WITH NUCLEIC PROBES, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

A “short hairpin RNA” or “small hairpin RNA” is a ribonucleotide sequence forming a hairpin turn which can be used to silence gene expression. After processing by cellular factors the short hairpin RNA interacts with a complementary RNA thereby interfering with the expression of the complementary RNA.

The words “protein”, “peptide”, and “polypeptide” are used interchangeably to denote an amino acid polymer or a set of two or more interacting or bound amino acid polymers.

A “dominant negative protein” is a modified form of a wild-type protein that adversely affects the function of the wild-type protein within the same cell. As a modified version of a wild-type protein the dominant negative protein may carry a mutation, a deletion, an insertion, a post-translational modification or combinations thereof. Any additional modifications of a nucleotide or polypeptide sequence known in the art are included. The dominant-negative protein may interact with the same cellular elements as the wild-type protein thereby blocking some or all aspects of its function.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “transfection” or “transfected” are defined by a process of introducing nucleic acid molecules into a cell by non-viral and viral-based methods. The nucleic acid molecules may be gene sequences encoding complete proteins or functional portions thereof.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

The term “reprogramming” refers to the process of dedifferentiating a non-pluripotent cell into a cell exhibiting pluripotent stem cell characteristics.

The term “treating” means ameliorating, suppressing, eradicating, and/or delaying the onset of the disease being treated.

### II. Methods of Preparing Induced Pluripotent Stem Cells

In one aspect, a method for preparing a method for preparing an induced pluripotent stem cell is provided. The method includes transfecting a non-pluripotent cell with a nucleic acid encoding an Oct4 protein, a nucleic acid encoding a Sox2 protein and a nucleic acid encoding a p53 inhibitor to form a transfected non-pluripotent cell. The transfected non-pluripotent cell is allowed to divide and thereby forms the induced pluripotent stem cell.

An “induced pluripotent stem cell” refers to a pluripotent stem cell artificially derived from a non-pluripotent cell. A non-pluripotent cell can be a cell of lesser potency to self-renew and differentiate than a pluripotent stem cell. Cells of lesser potency can be, but are not limited to somatic stem cells, tissue specific progenitor cells, primary or secondary cells. Without limitation, a somatic stem cell can be a hematopoietic stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

The term “transfection” or “transfecting” is defined as a process of introducing nucleic acid molecules into a cell by non-viral and viral-based methods. For non-viral methods of transfection any appropriate transfection method that does not use viral DNA or viral particles as a delivery system to introduce the nucleic acid molecule into the cell is useful in the methods described herein. Exemplary transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using retroviral vectors. In other embodiments, the nucleic acid molecules are introduced into a cell using lentiviral vectors.

An “Oct4 protein” as referred to herein includes any of the naturally-occurring forms of the Octomer 4 transcription factor, or variants thereof that maintain Oct4 transcription factor activity (e.g. within at least 50%, 80%, 90% or 100% activity compared to Oct4). In some embodiments, variants have at least 90% amino acid sequence identity across their whole sequence compared to the naturally occurring Oct4 polypeptide. In other embodiments, the Oct4 protein is the protein as identified by the NCBI reference gi:42560248 and gi:116235491 (isoforms 1 and 2).

A “Sox2 protein” as referred to herein includes any of the naturally-occurring forms of the Sox2 transcription factor, or variants thereof that maintain Sox2 transcription factor activity (e.g. within at least 50%, 80%, 90% or 100% activity compared to Sox2). In some embodiments, variants have at least 90% amino acid sequence identity across their whole sequence compared to the naturally occurring Sox2 polypeptide. In other embodiments, the Sox2 protein is the protein as identified by the NCBI reference gi:28195386.

A “p53 inhibitor” refers to a molecule that reduces p53 activity and expression. In some embodiments, the p53 inhibitor reduces the activity of a p53 protein. In other embodiments, the p53 inhibitor reduces the expression of a p53 gene. In some embodiments, the p53 inhibitor reduces the activity of a p53 protein and the expression of a p53 gene. Examples of a p53 inhibitor include, but are not limited to nucleic acids, proteins, dominant negative proteins, peptides, oligosaccharides, polysaccharides, lipids, phospholipids, glycolipids, monomers, polymers, small molecules and organic compounds. The p53 inibitor may be a polynucleotide. In some embodiments, the p53 inhibitor is a short hairpin RNA. In other embodiments, the p53 inhibitor is a small interfering RNA. The p53 inhibitor may be a protein. In some embodiments, the p53 inhibitor is a dominant negative protein.

Allowing the transfected non-pluripotent cell to divide and thereby forming the induced pluripotent stem cell may include expansion of the non-pluripotent cell after transfection, optional selection for transfected cells and identification of pluripotent stem cells. Expansion as used herein includes the production of progeny cells by a transfected non-pluripotent cell in containers and under conditions well know in the art. Expansion may occur in the presence of suitable media and cellular growth factors. Cellular growth factors are agents, which cause cells to migrate, differentiate, transform or mature and divide. They are polypeptides, which can usually be isolated from various normal and malignant mammalian cell types. Some growth factors can also be produced by genetically engineered microorganisms, such as bacteria (E. coli) and yeasts. Cellular growth factors may be supplemented to the media and/or may be provided through co-culture with irradiated embryonic fibroblast that secrete such cellular growth factors. Examples of cellular growth factors include, but are not limited to SCF, GMCSF, FGF, bFGF2, TNF, IFN, EGF, IGF and members of the interleukin family.

Where appropriate the expanding non-pluripotent cell may be subjected to a process of selection. A process of selection may include a selection marker introduced into a non-pluripotent cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected non-pluripotent cell the ability to expand in the presence of a toxin. Such a toxin typically inhibits cell expansion and/or causes cell death. Examples of such toxins include, but are not limited to hygromycin, neomycin, puromycin and gentamycin. In some embodiments, the toxin is hygromycin. Through the enzymatic activity of a selection maker a toxin may be converted to a non-toxin, which no longer inhibits expansion and causes cell death of a transfected non-pluripotent cell. Upon exposure to a toxin a cell lacking a selection marker may be eliminated and thereby precluded from expansion.

Identification of the induced pluripotent stem cell may include, but is not limited to the evaluation of the aforementioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

In some embodiments, the nucleic acid encoding an Oct4 protein forms part of a nucleic acid, the nucleic acid encoding a Sox2 protein forms part of a nucleic acid and the nucleic acid encoding a p53 inhibitor forms part of a nucleic acid. In another embodiment, the nucleic acid encoding an Oct4 protein, the nucleic acid encoding a Sox2 protein and the nucleic acid encoding a p53 inhibitor form part of the same nucleic acid. In other embodiments, the nucleic acid encoding an Oct4 protein and the nucleic acid encoding a Sox2 protein form part of a first nucleic acid and the nucleic acid encoding a p53 inhibitor form part of a second nucleic acid.

In some embodiments, the p53 inhibitor is a p53-specific short hairpin RNA. In other embodiments, the p53 inhibitor is a dominant negative p53 protein.

In another aspect, a method for preparing an induced pluripotent stem cell is provided. The method includes transfecting a p53-deficient non-pluripotent cell with a nucleic acid encoding an Oct4 protein and a nucleic acid encoding a Sox2 protein to form a transfected p53-deficient non-pluripotent cell. The transfected p53-deficient non-pluripotent cell is allowed to divide and thereby forms the induced pluripotent stem cell.

A “p53-deficient non-pluripotent cell” is a non-pluripotent cell that lacks p53 activity or expression. The lack of p53 activity and expression may be due to a genetic defect. The lack of p53 expression or activity may be due to a mutation, deletion or insertion in the p53 gene. The lack of p53 expression or activity may be due to the presence of a p53 inhibitor as aforementioned. In some embodiments, the p53-deficient non-pluripotent cell lacks p53 expression. In other embodiments, the p53-deficient non-pluripotent cell lacks p53 activity.

In another aspect, a method for preparing an induced pluripotent stem cell is provided. The method includes introducing a p53 inhibitor to a non-pluripotent cell. The non-pluripotent cell is transfected with a nucleic acid encoding an Oct4 protein and a nucleic acid encoding a Sox2 protein to form a transfected non-pluripotent cell. The transfection of the non-pluripotent cell is performed before, after or at the same time of introducing the p53 inhibitor to the non-pluripotent cell. The transfected non-pluripotent cell is allowed to divide and thereby forms the induced pluripotent stem cell. In some embodiments, transfecting the non-pluripotent cell is performed before introducing the p53 inhibitor to the non-pluripotent cell. In other embodiments, transfecting the non-pluripotent cell is performed after introducing the p53 inhibitor to the non-pluripotent cell. In other embodiments, transfecting the non-pluripotent cell is performed at the same time as introducing the p53 inhibitor to the non-pluripotent cell. Introducing a p53 inhibitor to the non-pluripotent cell includes administering the p53 inhibitor to the non-pluripotent cell by applying any useful methods known in the art. The p53 inhibitor may be administered to the non-pluripotent cell as a component of any suitable media or buffer. The p53 inhibitor may be administered to the non-pluripotent cell for a given time period and subsequently be removed. The p53 inhibitor may be administered to the non-pluripotent cell by microinjection.

In some embodiments, the p53 inhibitor is a chemical compound. In other embodiments, the p53 inhibitor is a small molecule.

In some embodiments, the non-pluripotent cell provided in the methods herein, is not transfected with an additional nucleic acid encoding a cMyc protein, a Lin28 protein, a Nanog protein or a Klf4 protein. The non-pluripotent cell may be transfected with a nucleic acid encoding an Oct4 protein, a nucleic acid encoding a Sox2 protein and a nucleic acid encoding a p53 inhibitor excluding additional nucleic acids encoding factors useful to generate an induced pluripotent stem cell from a non-pluripotent cell. In some embodiments, the p53-deficient non-pluripotent cell is transfected with a nucleic acid encoding an Oct4 protein and a nucleic acid encoding a Sox2 protein excluding additional nucleic acids encoding a cMyc protein, a Lin28 protein, a Nanog protein, a Klf4 protein or combinations thereof. In other embodiments, the non-pluripotent cell is introduced to a p53 inhibitor and transfected with a nucleic acid encoding an Oct4 protein and a nucleic acid encoding a Sox2 protein excluding additional nucleic acids encoding a cMyc protein, a Lin28 protein, a Nanog protein, a Klf4 protein or combinations thereof.

A “cMyc protein” as referred to herein includes any of the naturally-occurring forms of the cMyc transcription factor, or variants thereof that maintain cMyc transcription factor activity (e.g. within at least 50%, 80%, 90% or 100% activity compared to cMyc). In some embodiments, variants have at least 90% amino acid sequence identity across their whole sequence compared to the naturally occurring cMyc polypeptide. In other embodiments, the cMyc protein is the protein as identified by the NCBI reference gi:71774083.

A “Lin28 protein” as referred to herein includes any of the naturally-occurring forms of the Lin28 transcription factor, or variants thereof that maintain Lin28 transcription factor activity (e.g. within at least 50%, 80%, 90% or 100% activity compared to Lin28). In some embodiments, variants have at least 90% amino acid sequence identity across their whole sequence compared to the naturally occurring Lin28 polypeptide. In other embodiments, the Lin28 protein is the protein as identified by the NCBI reference gi:13375938.

A “Nanog protein” as referred to herein includes any of the naturally-occurring forms of the Nanog transcription factor, or variants thereof that maintain Nanog transcription factor activity (e.g. within at least 50%, 80%, 90% or 100% activity compared to Nanog). In some embodiments, variants have at least 90% amino acid sequence identity across their whole sequence compared to the naturally occurring Nanog polypeptide. In other embodiments, the Nanog protein is the protein as identified by the NCBI reference gi:153945816.

A “KLF4 protein” as referred to herein includes any of the naturally-occurring forms of the KLF4 transcription factor, or variants thereof that maintain KLF4 transcription factor activity (e.g. within at least 50%, 80%, 90% or 100% activity compared to KLF4). In some embodiments, variants have at least 90% amino acid sequence identity across their whole sequence compared to the naturally occurring KLF4 polypeptide. In other embodiments, the KLF4 protein is the protein as identified by the NCBI reference gi:194248077.

The methods for preparing induced pluripotent stem cells include that a nucleic acid encoding an Oct4 protein and a nucleic acid encoding a Sox2 protein are transfected into a non-pluripotent cell. In some embodiments, the nucleic acid encoding an Oct4 protein and the nucleic acid encoding a Sox2 protein form part of the same nucleic acid. In other embodiments, the non-pluripotent cell is a mammalian cell. In other embodiments, the non-pluripotent cell is a human cell. In some embodiments, the non-pluripotent cell is a mouse cell.

### III. An Induced Pluripotent Stem Cell

In one aspect, an induced pluripotent stem cell is prepared according to the methods provided herein.

### IV. Non-Pluripotent Cells

Provided herein are non-pluripotent cells useful as intermediates in making induced pluripotent stem cells.

In another aspect, a non-pluripotent cell including a nucleic acid encoding an Oct4 protein, a nucleic acid encoding a Sox2 protein and a nucleic acid encoding a p53 inhibitor is provided. In some embodiments, the non-pluripotent cell consists essentially of a nucleic acid encoding an Oct4 protein, a nucleic acid encoding a Sox2 protein and a nucleic acid encoding a p53 inhibitor. The non-pluripotent cell consisting essentially of a nucleic acid encoding an Oct4 protein, a nucleic acid encoding a Sox2 protein and a nucleic acid encoding a p53 inhibitor does not include any additional factors useful to generate an induced pluripotent stem cell from a non-pluripotent cell. The non-pluripotent cell consisting essentially of a nucleic acid encoding an Oct4 protein, a nucleic acid encoding a Sox2 protein and a nucleic acid encoding a p53 inhibitor does not include a nucleic acid encoding a Klf4 protein, a nucleic acid encoding a cMyc protein, a nucleic acid encoding a Nanog protein, a nucleic acid encoding a Lin28 protein or combinations thereof. In some embodiments, the nucleic acid encoding an Oct4 protein and the nucleic acid encoding a Sox2 protein form part of the same nucleic acid. In other embodiments, the nucleic acid encoding an Oct4 protein, the nucleic acid encoding a Sox2 protein and the nucleic acid encoding a p53 inhibitor form part of the same nucleic acid. In another embodiment, the nucleic acid encoding an Oct4 protein and the nucleic acid encoding a Sox2 protein form part of a first nucleic acid and the nucleic acid encoding a p53 inhibitor form part of a second nucleic acid.

In some embodiments, the non-pluripotent cell is a human cell. In another embodiment, the non-pluripotent cell is a mouse cell. In one embodiment, the p53 inhibitor is a p53-specific short hairpin RNA. In another embodiment, the p53 inhibitor is a dominant negative p53 protein.

In another aspect, a p53-deficient non-pluripotent cell including a nucleic acid encoding an Oct4 protein and a nucleic acid encoding a Sox2 protein is provided. In some embodiments, the p53-deficient non-pluripotent cell consists essentially of a nucleic acid encoding an Oct4 protein and a nucleic acid encoding a Sox2 protein. The p53-deficient non-pluripotent cell consisting essentially of a nucleic acid encoding an Oct4 protein and a nucleic acid encoding a Sox2 protein does not include any additional factors useful to generate an induced pluripotent stem cell from a non-pluripotent cell. The p53-deficient non-pluripotent cell consisting essentially of a nucleic acid encoding an Oct4 protein and a nucleic acid encoding a Sox2 protein does not include a nucleic acid encoding a Klf4 protein, a nucleic acid encoding a cMyc protein, a nucleic acid encoding a Nanog protein, a nucleic acid encoding a Lin28 protein or combinations thereof.

In another aspect, a non-pluripotent cell including a nucleic acid encoding an Oct4 protein, a nucleic acid encoding a Sox2 protein and a p53 inhibitor is provided. In some embodiments, the non-pluripotent cell consists essentially of a nucleic acid encoding an Oct4 protein, a nucleic acid encoding a Sox2 protein and a p53 inhibitor. The non-pluripotent cell consisting essentially of a nucleic acid encoding an Oct4 protein, a nucleic acid encoding a Sox2 protein and a p53 inhibitor does not include any additional factors useful to generate an induced pluripotent stem cell from a non-pluripotent cell. The non-pluripotent cell consisting essentially of a nucleic acid encoding an Oct4 protein, a nucleic acid encoding a Sox2 and a p53 inhibitor does not include a nucleic acid encoding a Klf4 protein, a nucleic acid encoding a cMyc protein, a nucleic acid encoding a Nanog protein, a nucleic acid encoding a Lin28 protein or combinations thereof.

### V. Methods for Producing Human Somatic Cells from Induced Pluripotent Stem Cells

In one aspect, a method of treating a mammal in need of tissue repair is provided. The method includes administering an induced pluripotent stem cell to the mammal. The induced pluripotent stem cell is allowed to divide and differentiate into somatic cells in the mammal thereby providing tissue repair in the mammal. The induced pluripotent stem cell may be prepared by a process that includes transfecting a non-pluripotent cell with a nucleic acid encoding an Oct4 protein, a nucleic acid encoding a Sox2 protein and a nucleic acid encoding a p53 inhibitor to form a transfected non-pluripotent cell. The transfected non-pluripotent cell is allowed to divide and thereby forms the induced pluripotent stem cell. The induced pluripotent stem cell may be prepared by a process that includes transfecting a p53-deficient non-pluripotent cell with a nucleic acid encoding an Oct4 protein, a nucleic acid encoding a Sox2 protein to form a transfected p53-deficient non-pluripotent cell. The transfected p53-deficient non-pluripotent cell is allowed to divide and thereby forms the induced pluripotent stem cell. The induced pluripotent stem cell may be prepared by a process that includes introducing a p53 inhibitor to a non-pluripotent cell. The non-pluripotent cell is transfected with a nucleic acid encoding an Oct4 protein and a nucleic acid encoding a Sox2 protein to form a transfected non-pluripotent cell. The transfection of the non-pluripotent cell is performed before, after or at the same time of introducing the p53 inhibitor to the non-pluripotent cell. The transfected non-pluripotent cell is allowed to divide and thereby forms the induced pluripotent stem cell.

In another aspect, a method for producing a somatic cell is provided. The method includes contacting an induced pluripotent stem cell with a cellular growth factor. The induced pluripotent stem cell is allowed to divide, thereby forming the somatic cell. The induced pluripotent stem cell may be prepared by a process that includes transfecting a non-pluripotent cell with a nucleic acid encoding an Oct4 protein, a nucleic acid encoding a Sox2 protein and a nucleic acid encoding a p53 inhibitor to form a transfected non-pluripotent cell. The transfected non-pluripotent cell is allowed to divide and thereby forms the induced pluripotent stem cell. The induced pluripotent stem cell may be prepared by a process that includes transfecting a p53-deficient non-pluripotent cell with a nucleic acid encoding an Oct4 protein, a nucleic acid encoding a Sox2 protein to form a transfected p53-deficient non-pluripotent cell. The transfected p53-deficient non-pluripotent cell is allowed to divide and thereby forms the induced pluripotent stem cell. The induced pluripotent stem cell may be prepared by a process that includes introducing a p53 inhibitor to a non-pluripotent cell. The non-pluripotent cell is transfected with a nucleic acid encoding an Oct4 protein and a nucleic acid encoding a Sox2 protein to form a transfected non-pluripotent cell. The transfection of the non-pluripotent cell is performed before, after or at the same time of introducing the p53 inhibitor to the non-pluripotent cell. The transfected non-pluripotent cell is allowed to divide and thereby forms the induced pluripotent stem cell.

### EXAMPLES

The following data reveal that the p53 pathway is activated in response to signaling associated with reprogramming. Reducing signaling to p53 through expression of mutant forms of one of its negative regulators or deleting or silencing p53 or its target gene, p21, or antagonizing apoptosis enhanced three-factor (Oct4/Sox2/Klf4)-mediated reprogramming of mouse fibroblasts. Notably, decreasing p53 protein levels enabled fibroblasts to give rise to iPS cells capable of generating chimeric mice using only Oct4 and Sox2. Furthermore, silencing of p53 significantly increased the reprogramming efficiency of human somatic cells. The present findings provide insights into reprogramming mechanisms and suggest new routes to more efficient reprogramming while minimizing the use of oncogenes.

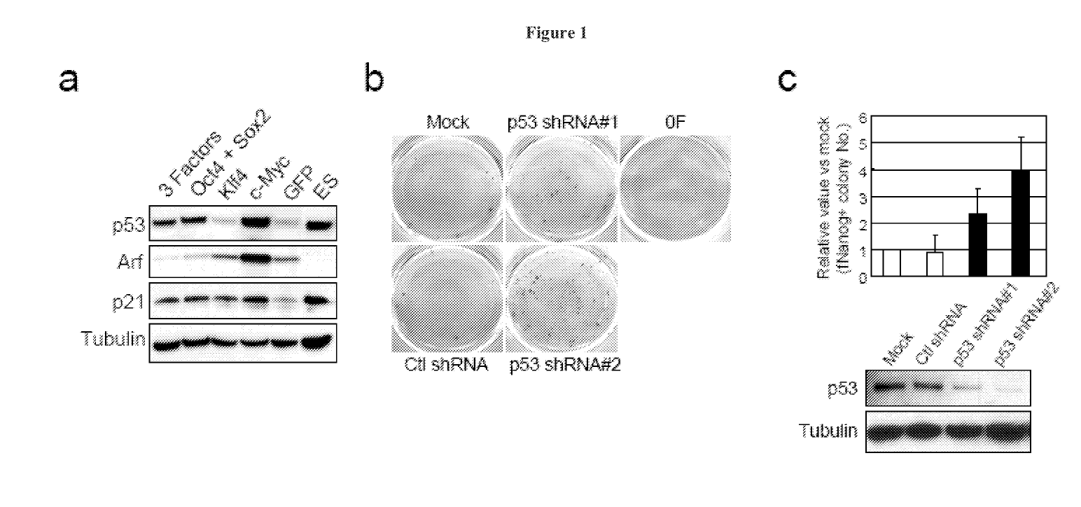

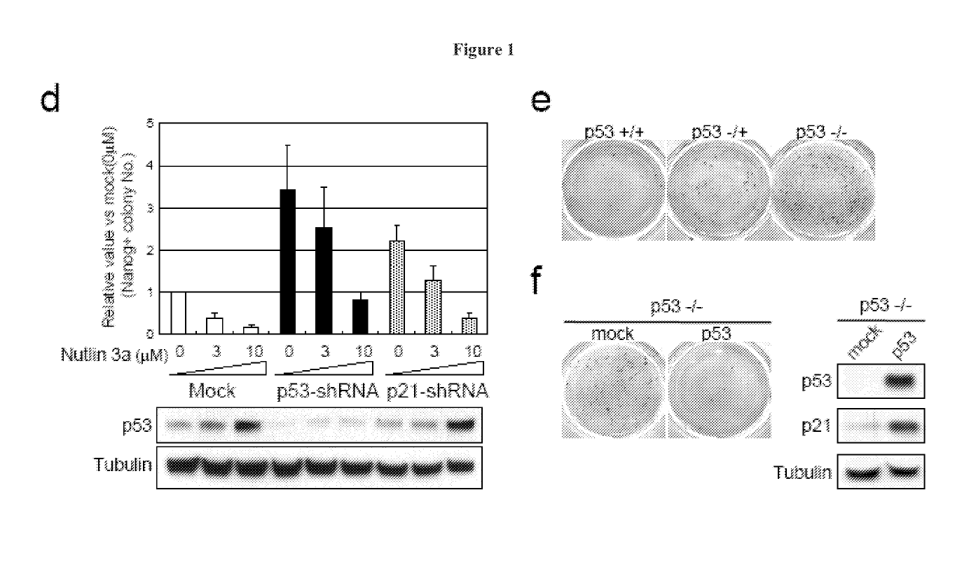

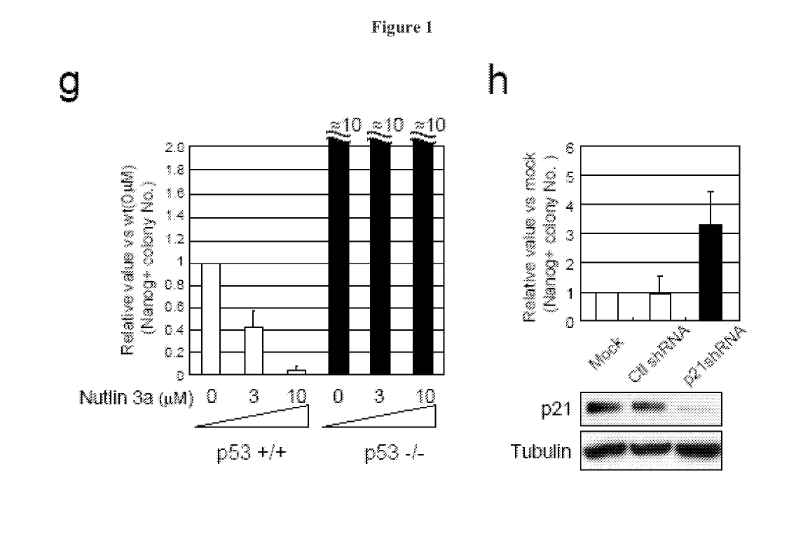

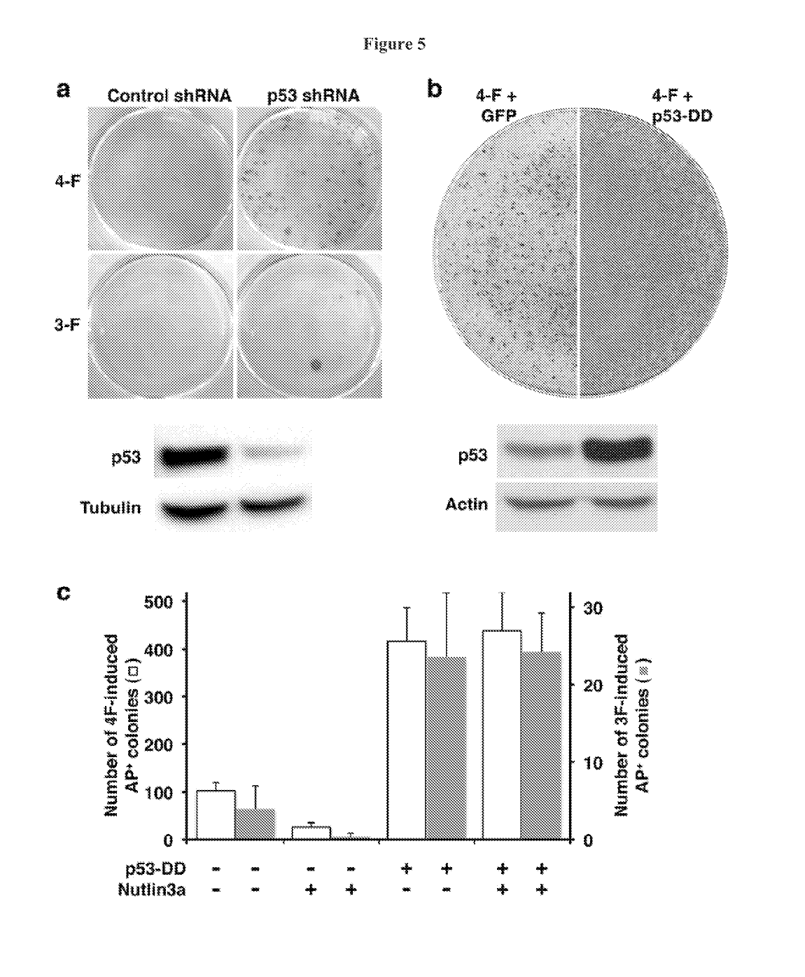

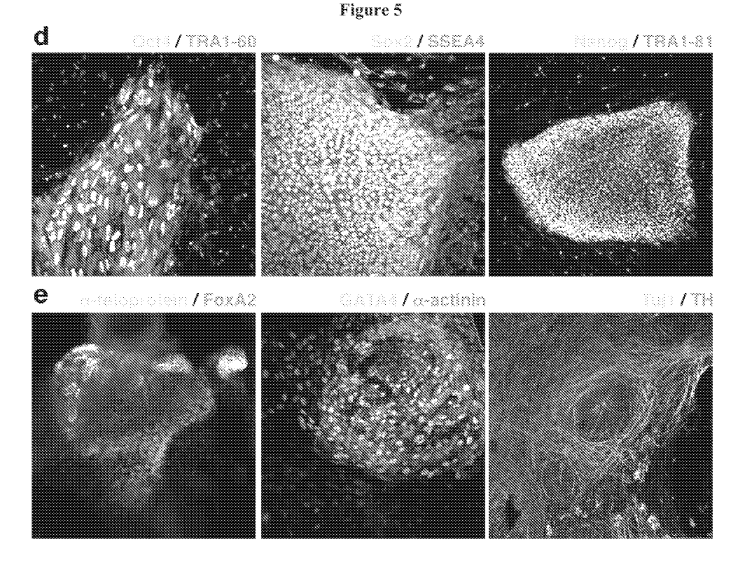

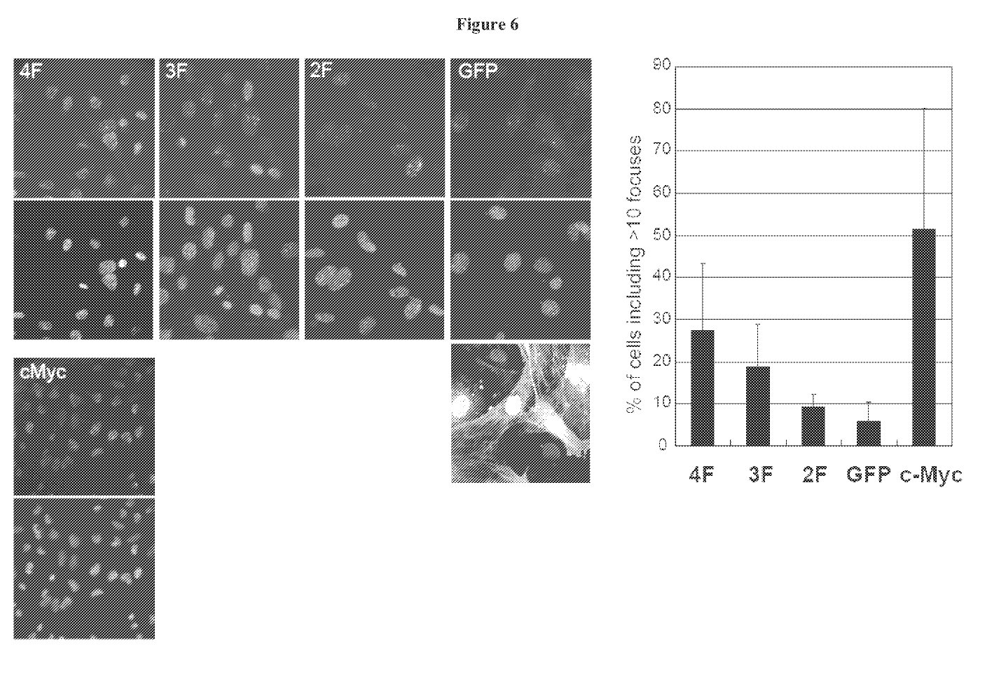

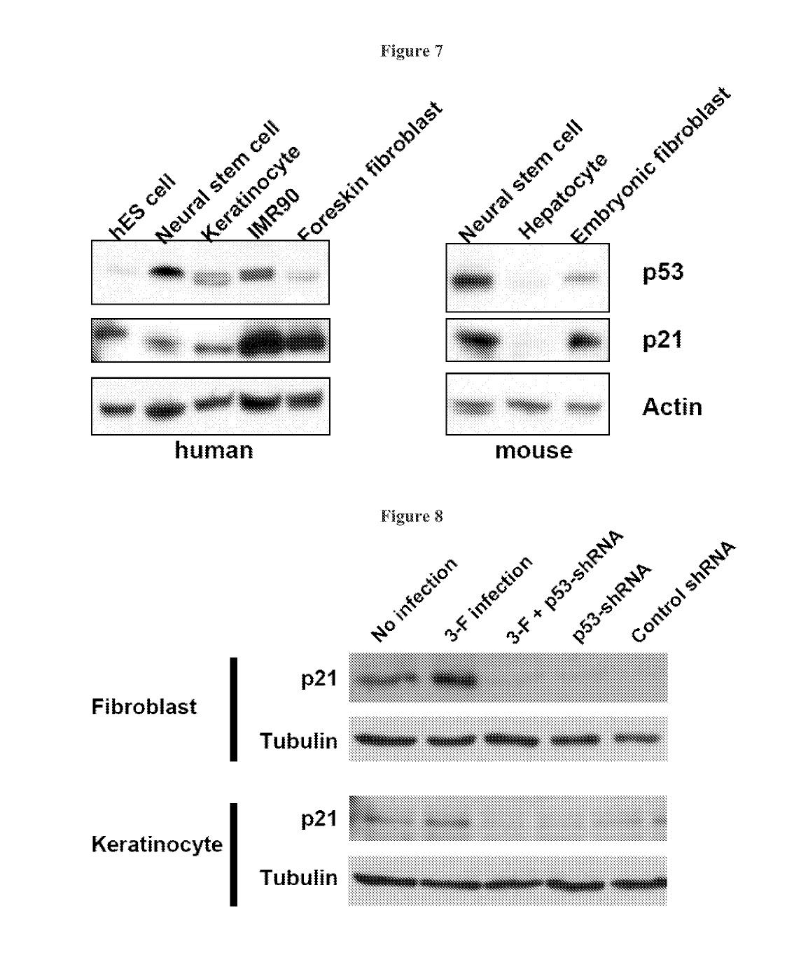

It was first determined whether the reprogramming factors, individually or in combination, activate the p53 pathway in mouse embryo fibroblasts (MEFs). Relative to the GFP-retroviral control, c-Myc significantly increased p53 abundance and activity, manifested by increased expression of the cyclin-dependent kinase inhibitor p21 (FIG. 1a). This was achieved by induction of Arf, an antagonist of Mdm2, the E3-ubiquitin ligase responsible for p53 degradation¹². Modest changes in activation of the p53 target gene p21, with or without changes in p53 abundance, were noted in response to infection with viruses encoding Klf4, Oct4/Sox2 (2-F) or Oct4/Sox2/Klf4 (3-F) (FIG. 1a). Since introduction of reprogramming factors increased γ-H2AX foci, it was speculated that their expression induces p53 via DNA damage. See FIG. 6. Also, p53 and p21 expression was compared in a variety of mouse and human cell lines previously utilized for iPS cell production. See FIG. 7. Interestingly, keratinocytes display lower p53 and p21 protein levels than other cell types. Furthermore, infection with 3-F resulted in higher p21 induction in fibroblasts than in keratinocytes. See FIG. 8. These data are consistent with p53 pathway activation by the conditions used for human fibroblast reprogramming, which occurs with extremely low efficiency. The data further imply that the higher reprogramming efficiency of human keratinocytes may derive from their lower levels and reduced activation of p53 and p21.

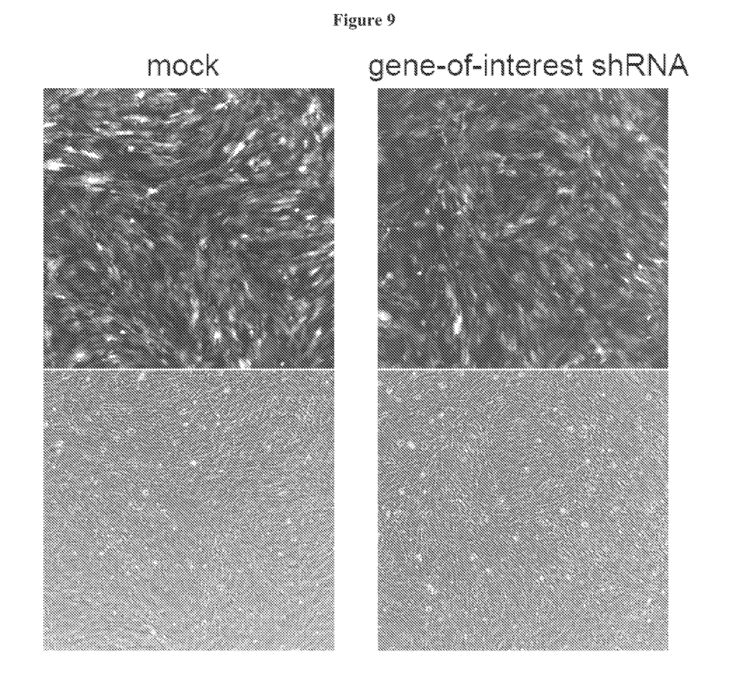

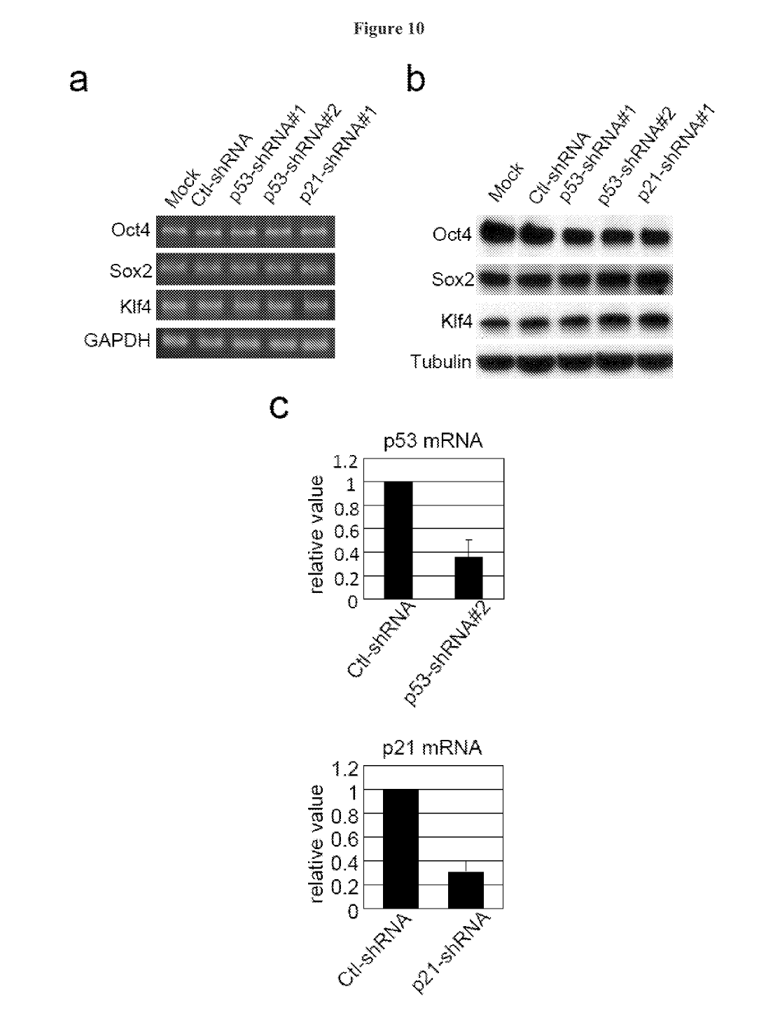

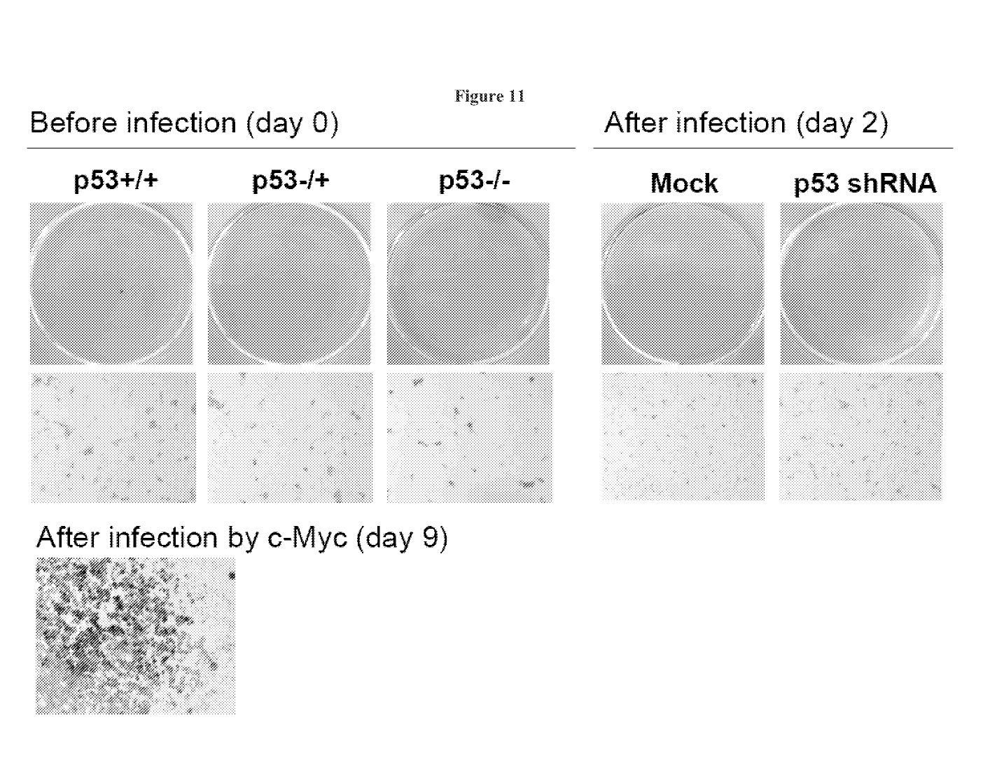

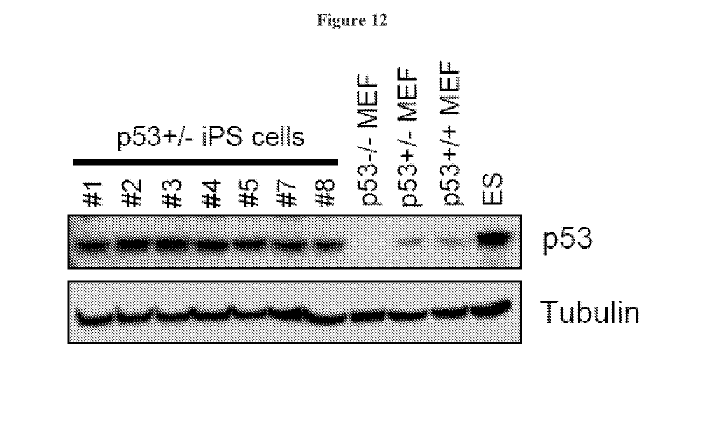

If p53 pathway activation by 3-F lowers reprogramming efficiency, then eliminating p53 should increase the frequency of obtaining iPS cells. This was investigated by both reducing p53 expression using short-hairpin RNA (shRNA) in MEFs as well as by eliminating all p53 using p53-null MEFs. Under the infection conditions employed, most cells were infected (FIG. 9) and p53 mRNA and protein levels were reduced by 60-80% (FIG. 1c and FIG. 10). In MEFs, infection with 3-F increased colony formation by 2-4-fold using two different shRNAs (FIGS. 1b, c). This is likely to be an under-estimate of the magnitude of the p53 suppressive capacity, since p53 protein clearly remained present. In agreement with this, MEFs in which p53 was reduced using the most effective shRNA were treated with the p53 agonist Nutlin3a, which selectively activates p53 by preventing Mdm2 binding¹³. Nutlin3a treatment resulted in a dose-dependent decrease in iPS cell formation (FIG. 1d). By contrast, p53-null MEFs increased colony formation by at least 10-fold (Table 1; FIG. 1e, FIG. 1g), and in this context Nutlin3a treatment did not have any effects (FIG. 1g). p53+/− heterozygous MEFs also allowed formation of more 3-F induced clones than wild-type MEFs. While culture stress can induce cellular senescence and activate p53, which would reduce reprogramming, fewer than 1% of the cells of all p53 genotypes stained with the senescence marker β-galactosidase. See FIG. 11. Since a loss of heterozygosity of the p53 gene in iPS colonies derived from p53+/− MEF was not detected (FIG. 12), the data suggest a p53 dosage-sensitivity to reprogramming (FIG. 1e). Since p53 null MEFs are genetically unstable, increased reprogramming efficiency might result from expression of the 3-F in variant cells. However, it was found that re-expressing p53 in the p53-null MEFs dramatically reduced reprogramming efficiency (FIG. 1f).

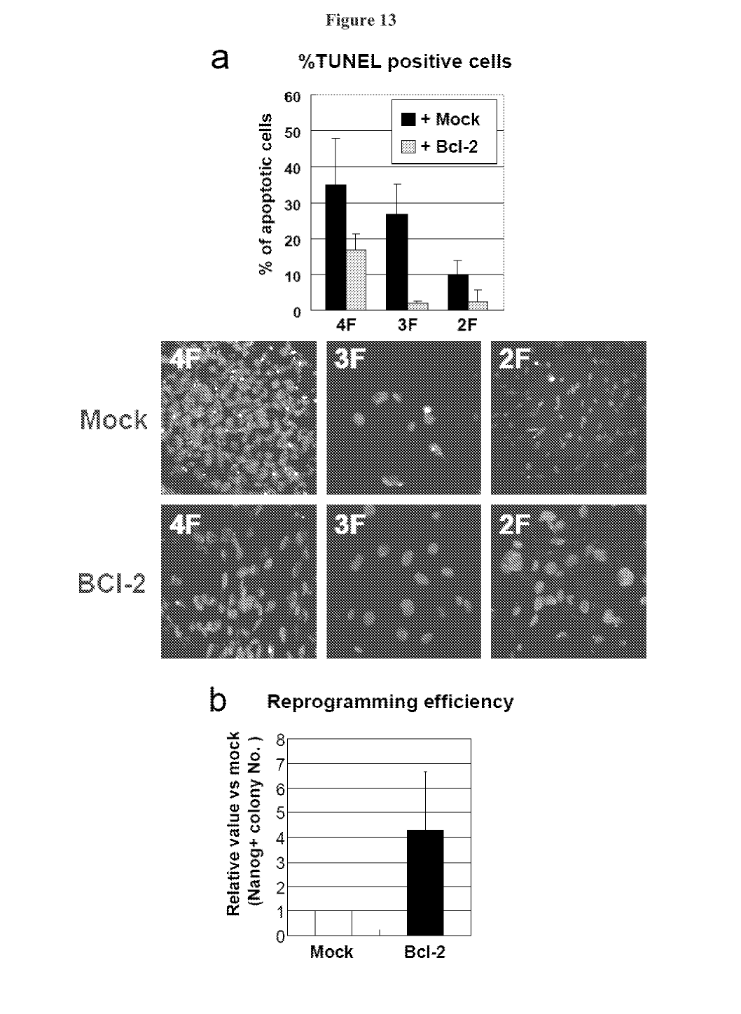

These data indicate that it is the activity of p53, rather than the genomic consequences of its loss, that reduce reprogramming efficiency. This was investigated further by determining whether p53-induced gene products also affect reprogramming efficiency. Since p21 was induced in response to 3-F expression, p21 shRNA expressing lentiviruses were used to reduce p21 expression and reprogramming efficiency was then determined. An approximately 3-fold increase in reprogramming (FIG. 1h) was observed. However, the p21 shRNA only reduced p21 expression by approximately 3-4 fold, and was not able to overcome conditions that result in p21 induction, such as Nutlin3a treatment (FIG. 1d). Thus, the modest increase in reprogramming efficiency in p21 shRNA expressing cells is likely an underestimate of the true extent to which p21 induction suppresses reprogramming. A modest induction of the pro-apoptotic factor BAX, a p53-inducible gene¹⁴ in 3-F experiments (data not shown) was also noted. Consistent with a limiting role of the p53 induced apoptotic response during reprogramming, overexpression of the BAX antagonist Bcl-2 suppressed apoptosis in 2-F, 3-F and 4-F experiments and increased the frequency of colonies expressing the pluripotency factor Nanog by 4-fold. See FIG. 13.

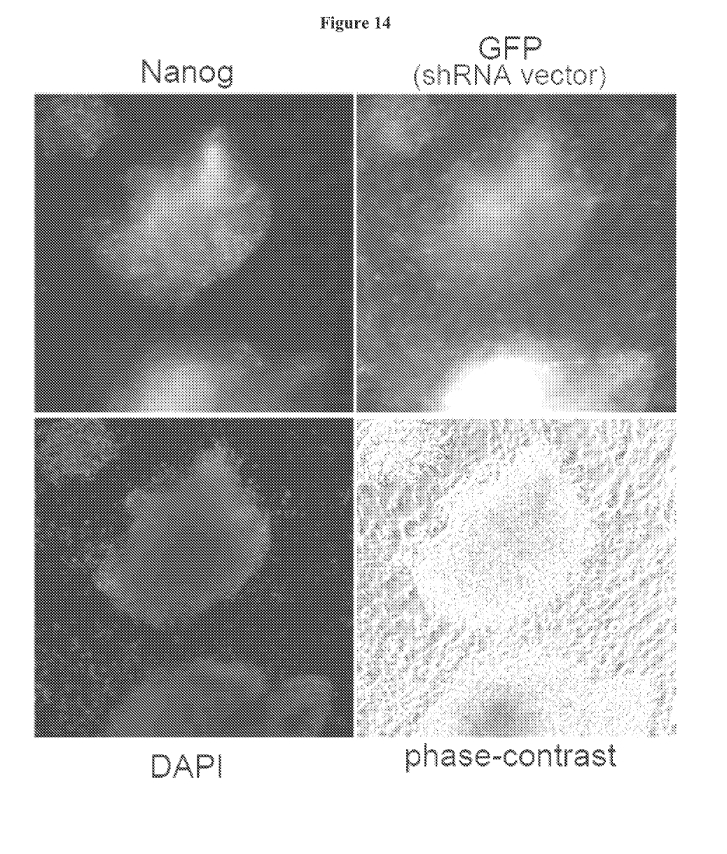

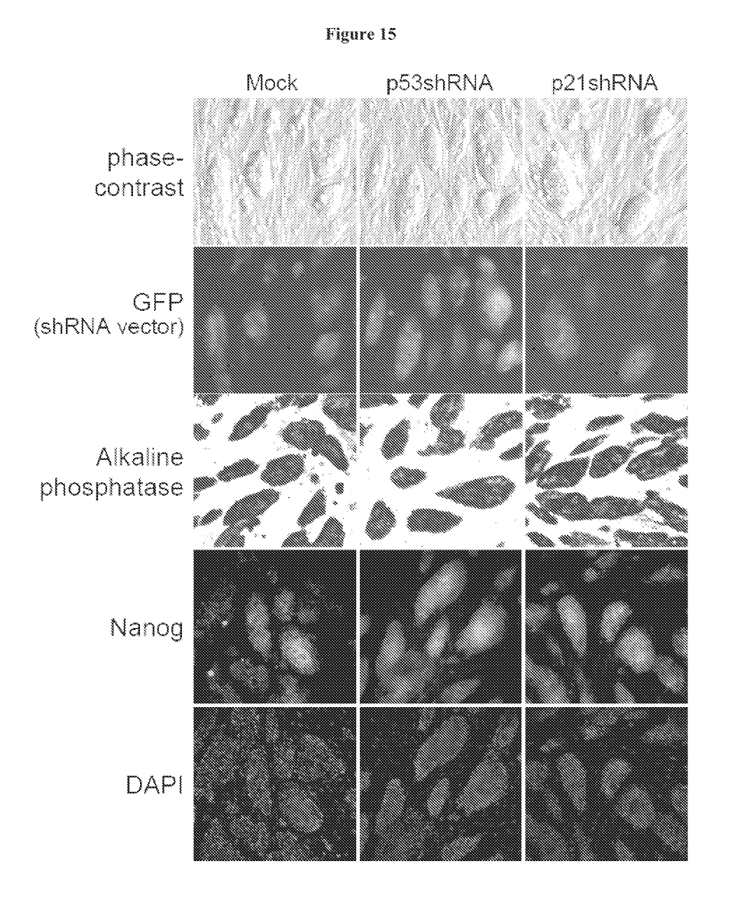

The 3-F transduced colonies exhibit the characteristics of iPS cells. See FIGS. 14-16. First, they displayed typical mouse ES cell-like morphology and stained strongly positive for alkaline phosphatase activity. Second, they expressed transcription factors and cell surface markers characteristic of mouse ES cells, including Nanog. Third, they readily differentiated into derivatives of all three embryonic germ layers in vitro. Taken together with the genetic and shRNA studies described above, these data show that complete loss of p53 function dramatically increases reprogramming efficiency, and that even low levels of p53 activity are all that is needed to compromise reprogramming of somatic cells.

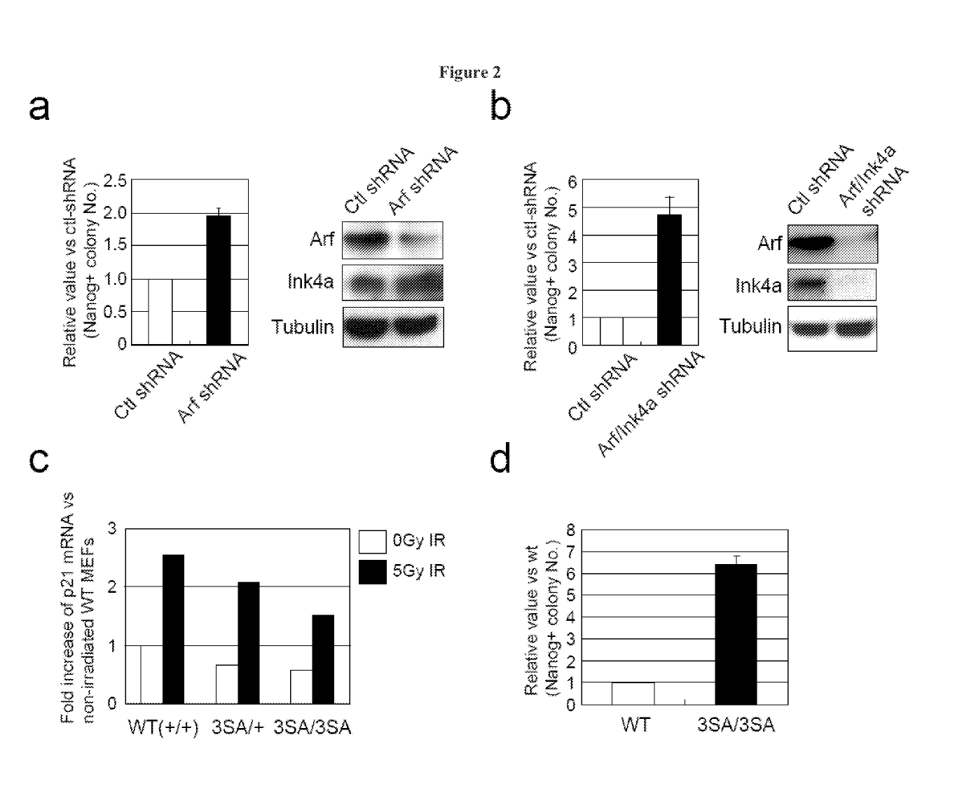

The ability of the 3-F to increase p53 abundance suggests that controlling its stability might be crucial for p53-mediated reprogramming suppression. It was initially determined whether reducing Arf levels using Arf shRNA increases reprogramming efficiency, as lower Arf levels should decrease p53 stability¹⁵⁻¹⁷. Arf shRNA reduced Arf levels by 2-4 fold (FIG. 2a). This level of Arf reduction enabled an increase of 3-F reprogramming by approximately 2-fold (FIG. 2a). Since p21 reduction increased reprogramming, and p21 expression prevents cell cycle progression by antagonizing cyclin dependent kinases that are required to inactivate the retinoblastoma (Rb) tumor suppressor¹⁸, it was investigated whether antagonizing Rb function would increase iPS cell formation. Rb function was indirectly compromised by reducing the expression of the cyclin dependent kinase inhibitor p16Ink4a, which inhibits the cdk4-mediated phosphorylation of Rb that contributes to its inactivation¹⁸. It was found that reducing Arf and p16Ink4a together increased iPS cell formation 4-5 fold, which was more than Arf reduction alone (FIG. 2b). These data indicate that reducing p53 activation by antagonizing Arf, and reducing Rb repression by antagonizing p16Ink4a, collaborate to improve reprogramming efficiency.

Next the activity of the E3 ligase that regulates p53 stability was genetically modulated by generating a mouse encoding a mutant version of Mdm×¹⁹, a RING domain heterodimeric partner of Mdm2²⁰. Regulating the stability of the Mdm2/Mdmx heterodimer is critical for p53 activation²⁰. For example, DNA damage or activated oncogenes result in phosphorylation of multiple serine residues in Mdmx, which leads to its accelerated degradation²⁰. Substituting three serines by alanines (Mdmx S342A, S367A, 5403A, hereafter called Mdmx3SA) significantly stabilizes Mdmx to DNA damage in vitro²⁰, and in mice encoding Mdmx3SA. Importantly, MEFs or thymocytes derived from homozygous Mdmx3SA mice exhibited lower basal expression of p21 as well as lower DNA damage induced p21 levels (FIG. 2c, and ref 19). Mdmx3SA also significantly impaired the ability of c-Myc to activate p53 in vivo¹⁹. As Mdmx3SA MEFs are less sensitive to signals elicited by DNA damage and by activated oncogenes, it was determined if they undergo reprogramming at a higher efficiency than wild-type MEFs. The results (FIG. 2d) show that 3-F reprogramming is increased ˜7 fold in Mdmx3SA MEFs. The 3SA mutations are in serines targeted by damage kinases including ATM and Chk2²⁰, and the data shown above indicate that DNA damage can result from introduction of reprogramming factors. However, it is also possible that DNA damage is not involved, or not the only factor that activates p53 during reprogramming, since kinases such as ATM may also be activated by alterations in chromatin structure²¹, a known requirement for reprogramming.

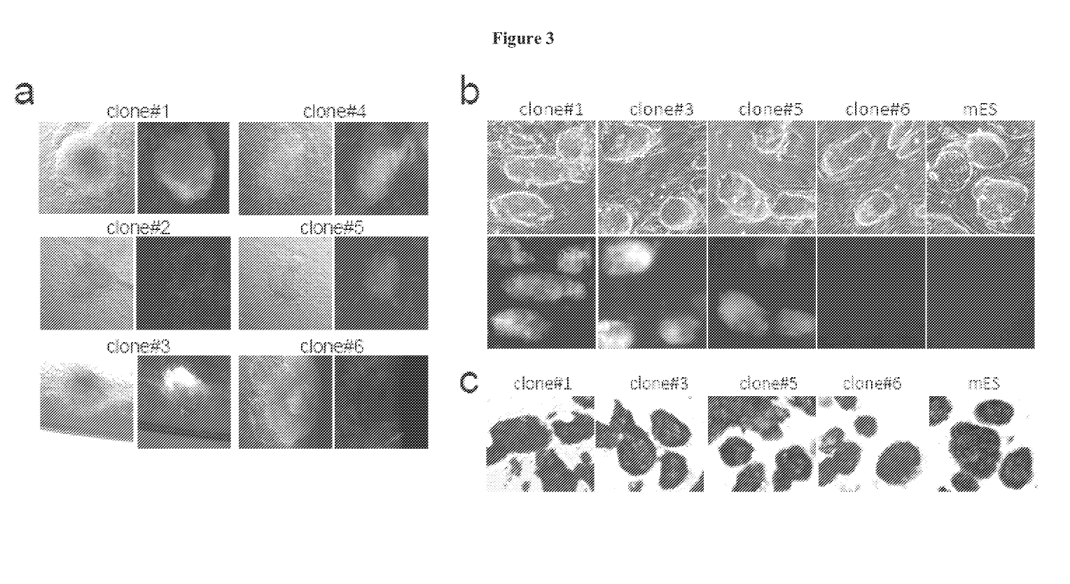

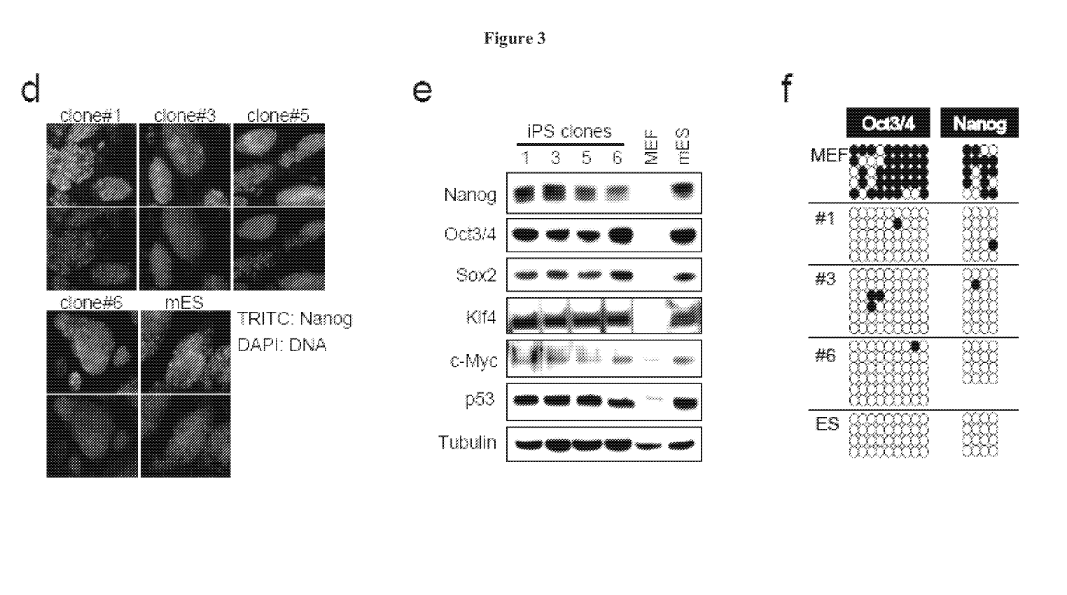

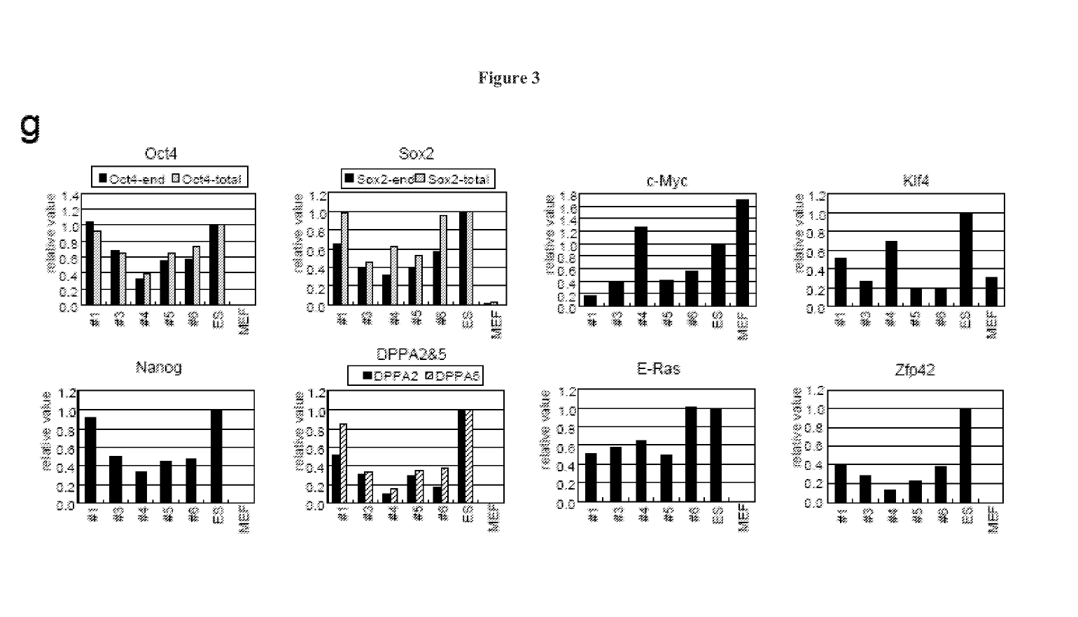

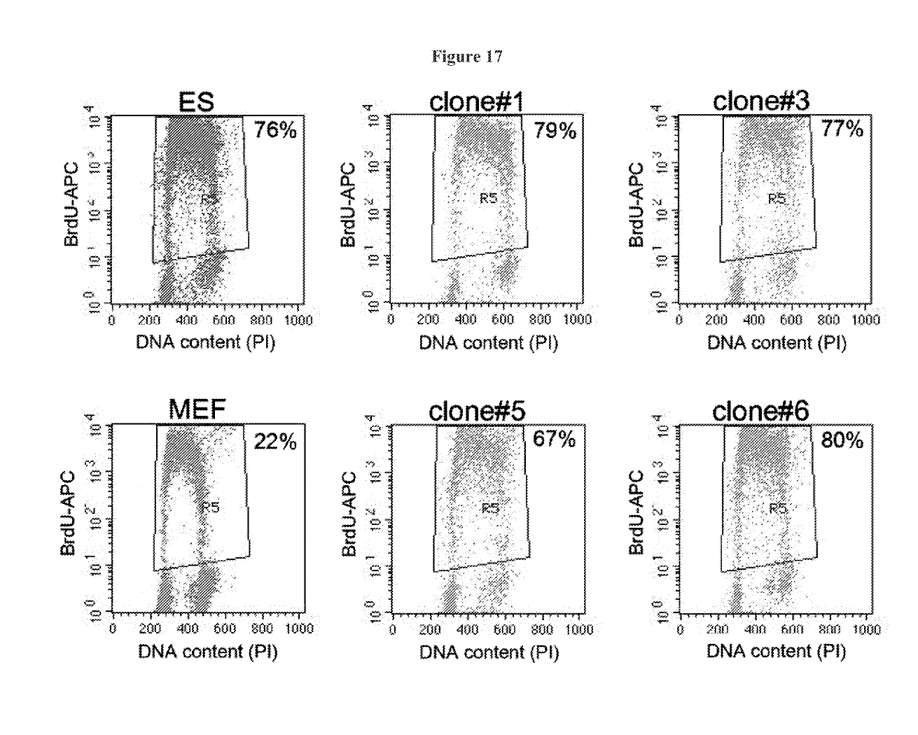

Clinical application of reprogramming requires elimination of the oncogenes to limit malignant transformation. The generation of iPS cells in the absence of c-Myc in cells with reduced p53 expression as reported above is one step towards achieving that goal. However, as Klf4 has also been reported to have oncogenic properties when overexpressed²², and it has been shown that it alone can activate p53, it was investigated whether cells with reduced p53 expression could be converted into iPS cells using only two factors, Oct4, and Sox2. This hypothesis was tested by transducing MEFs with a lentivirus expressing p53 shRNA plus retroviruses encoding Oct4 and Sox2 (hereafter designated as 2F-p53KD-iPS cells). Cells that developed into colonies exhibiting ES cell-like morphology were obtained by week four post-infection. Six colonies were selected for further analysis (FIG. 3a) and four could be maintained by standard procedures for the culture of mouse ES cells (FIG. 3b). These four clones stained positive for alkaline phosphatase activity (FIG. 3c) and expressed genes and cell surface markers characteristic of mouse ES cells including the pluripotency marker Nanog (FIGS. 3d, e). Overall, the expression of stem cell markers in 2F-p53KD-iPS cells was indistinguishable from that of mouse ES cell lines maintained under similar conditions. Bisulfite sequencing of the Oct4 and Nanog promoters revealed nearly complete demethylation in 2F-p53KD-iPS cells when compared to MEFs (FIG. 3f). Consistent with previous reports, where generation of iPS cells requires silencing of the retroviral genes, it was found that transgene silencing occurred in all the 2F-p53KD-iPS colonies (FIG. 3g). Also, like ES cells, the majority (70-80%) of cells were in S-phase. See FIG. 17.

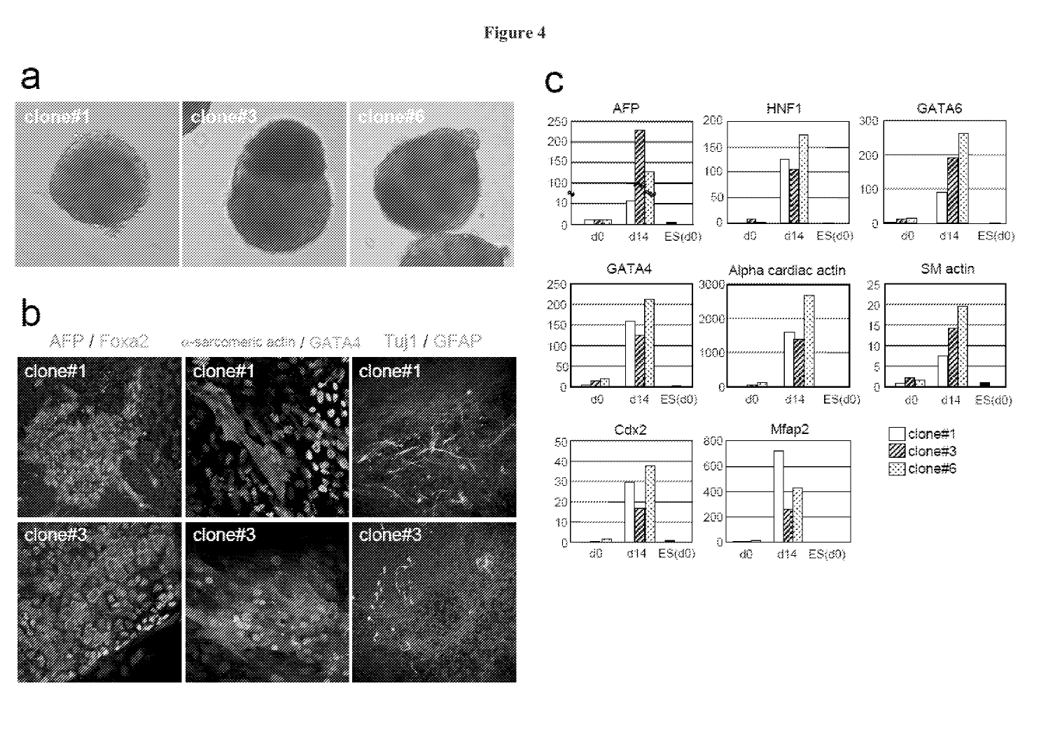

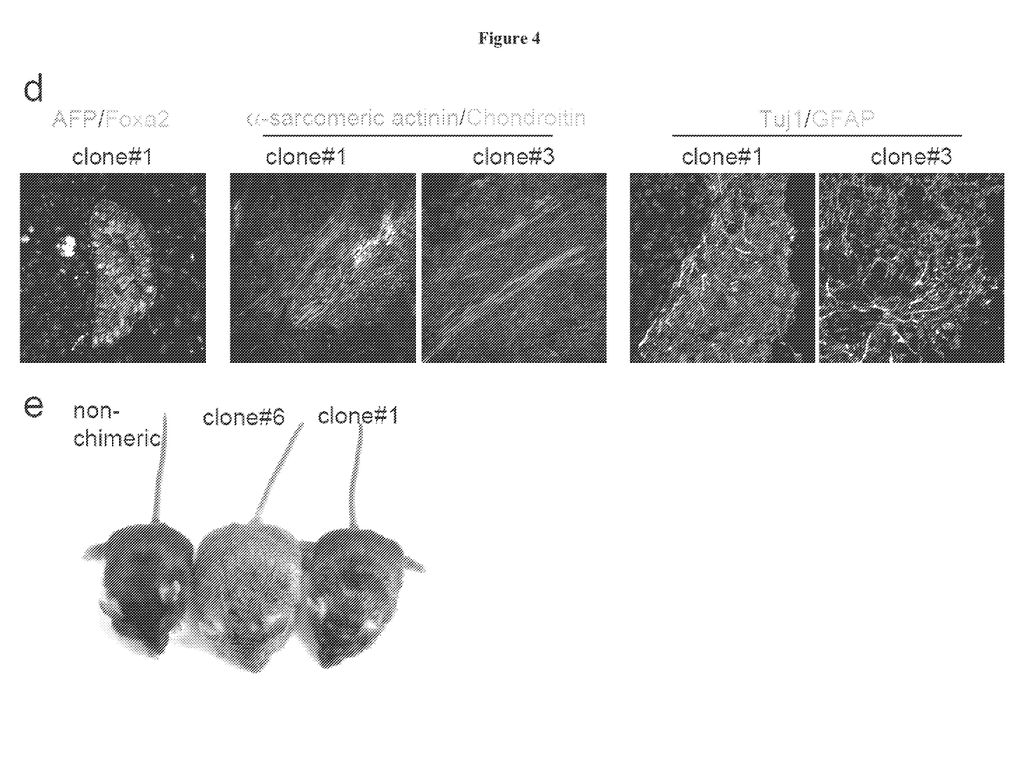

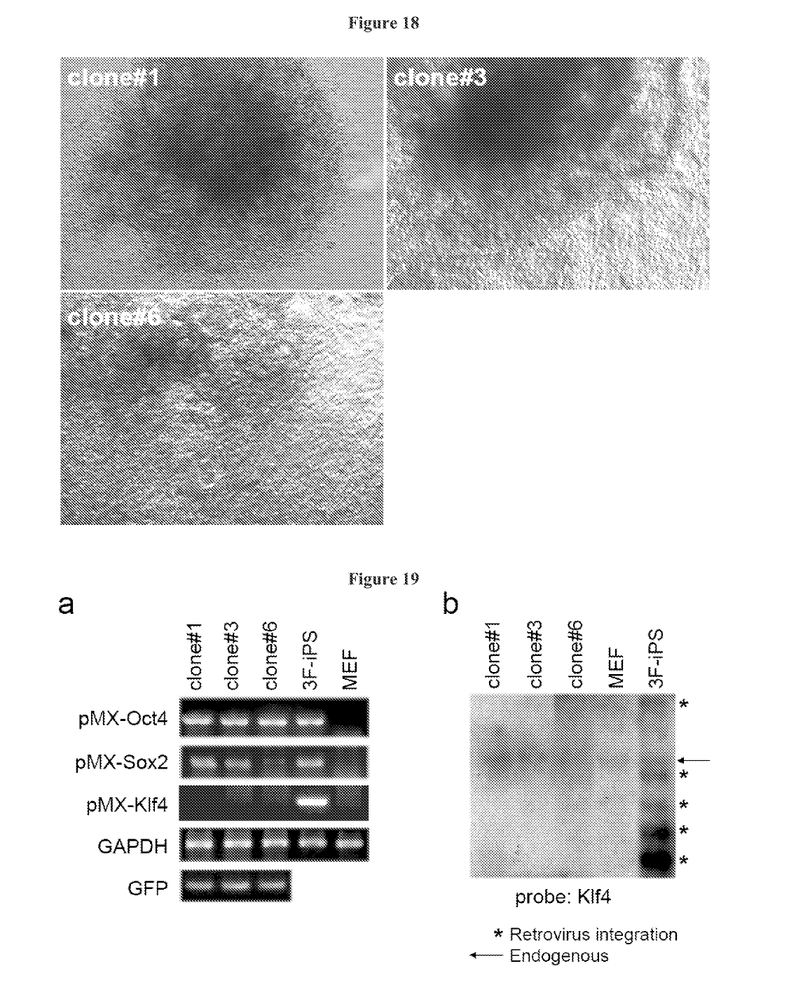

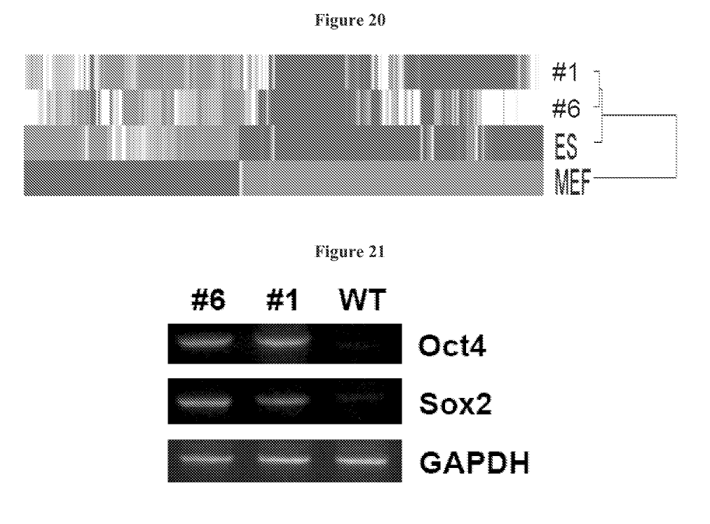

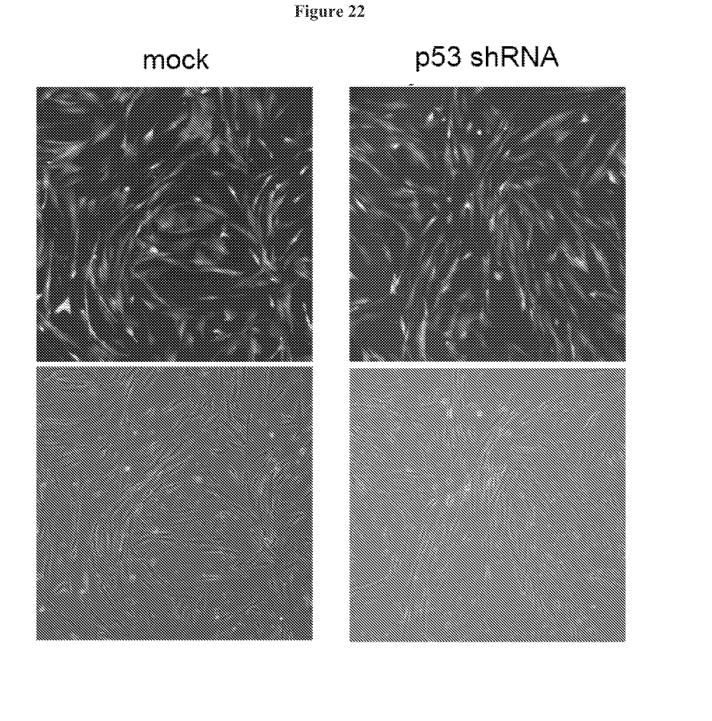

The pluripotency of three 2F-p53KD-iPS clones was tested in assays of embryoid body formation in vitro (FIG. 4a) and/or teratoma induction in vivo (FIG. 4d). The tested cell lines differentiated into the three germ layer derivatives, as shown by α-fetoprotein(AFP)/Foxa2 (endoderm), α-sarcomeric actin/GATA4 (mesoderm) and Tuj1/GFAP (ectoderm) immunostaining and mRNA expression (FIGS. 4b, c). Furthermore, these cells differentiated with high efficiency into beating cardiomyocytes. See FIG. 18. Upon injection into immunocompromised mice, two independent 2F-p53KD-iPS lines generated complex intratesticular and subcutaneous teratomas containing structures and tissues representative of the three embryonic germ layers (FIG. 4d). To further characterize these iPS cell clones, microarray analysis was performed, confirming that gene expression patterns of these clones are similar to mouse ES cells. See FIG. 20. Finally, 2F-p53KD-iPS were able to contribute to chimeric mice when injected into mouse blastocysts. One line (clone#6) contributed almost 100% to chimera formation, and the other line (clone#1) contributed 30-50%, as judged by coat color (FIG. 4e, FIG. 21). Taken together, these results demonstrate that MEFs can be reprogrammed to pluripotency by the forced expression of only two factors, Oct4 and Sox2, when p53 levels are reduced.

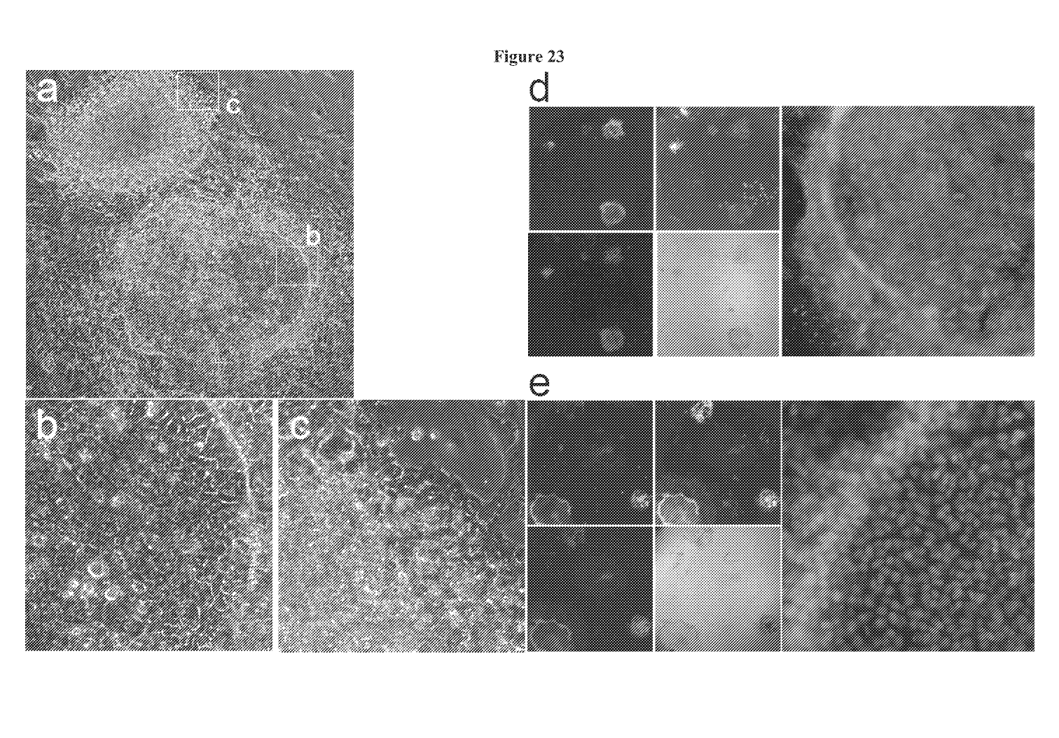

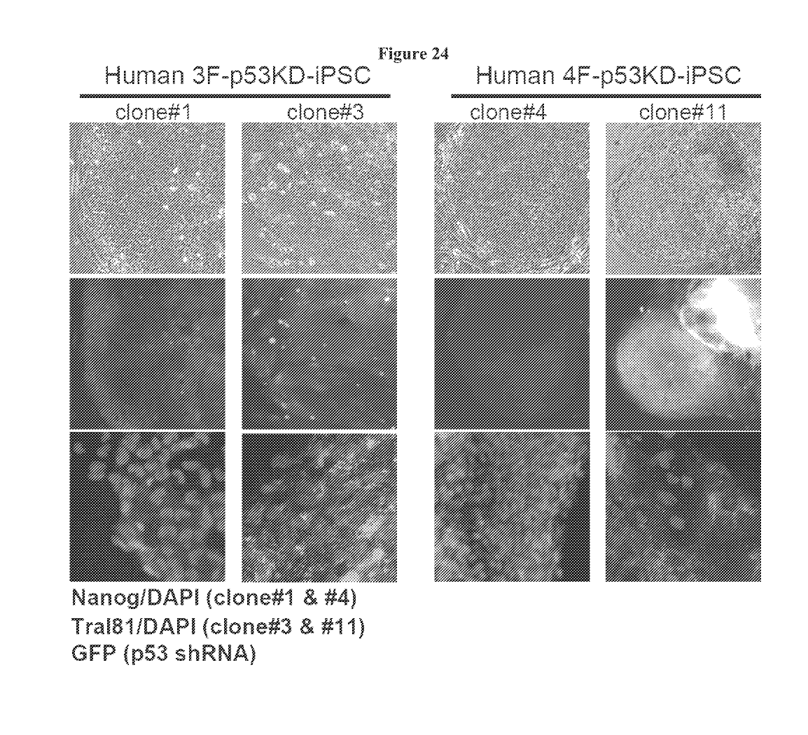

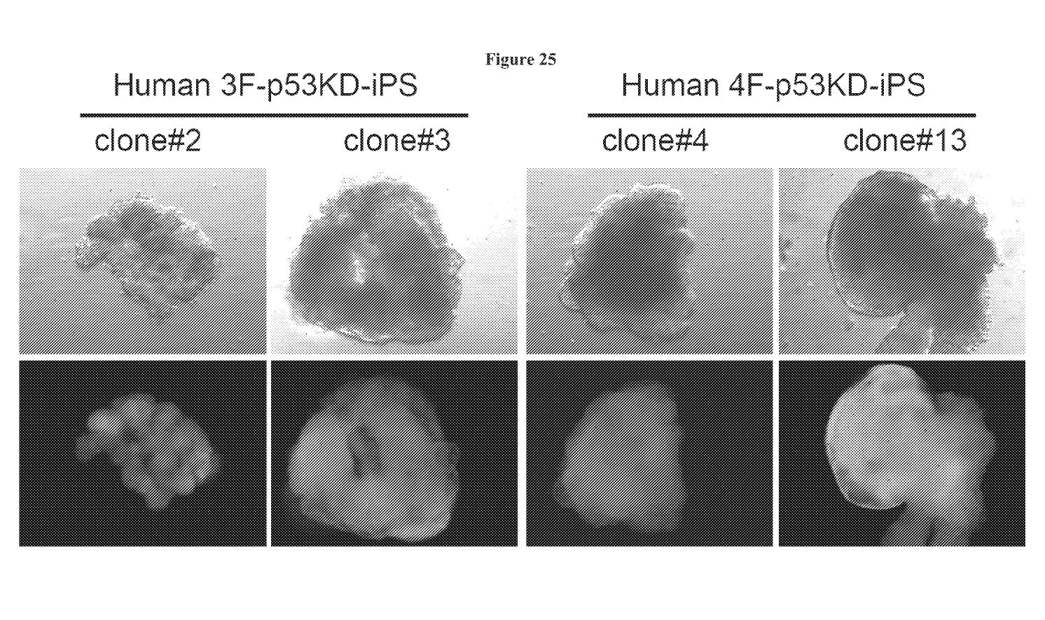

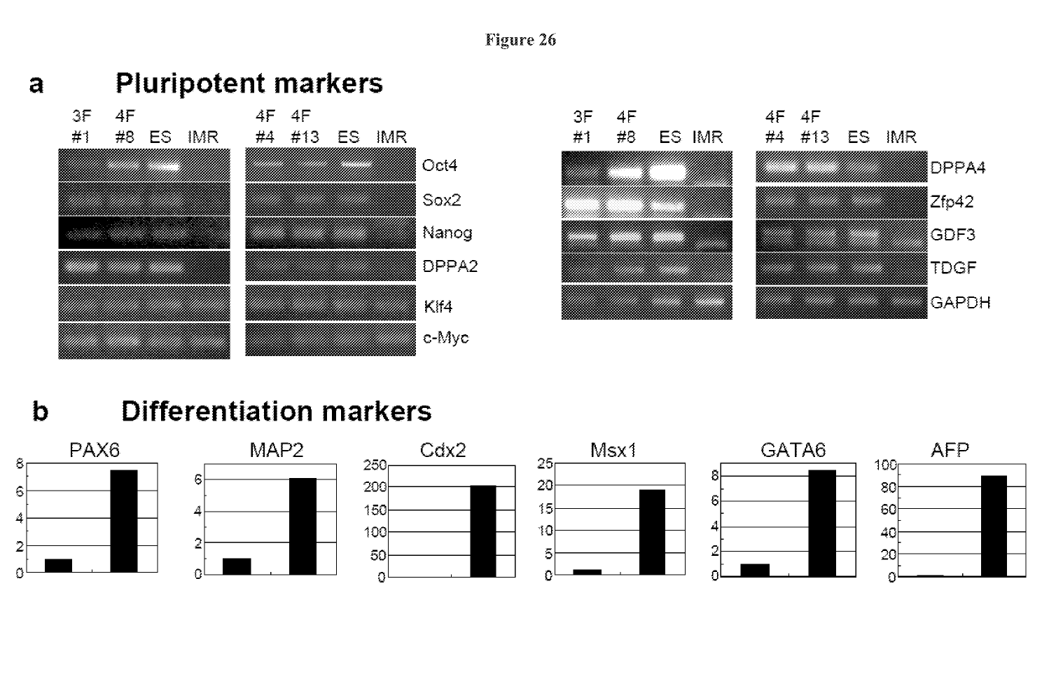

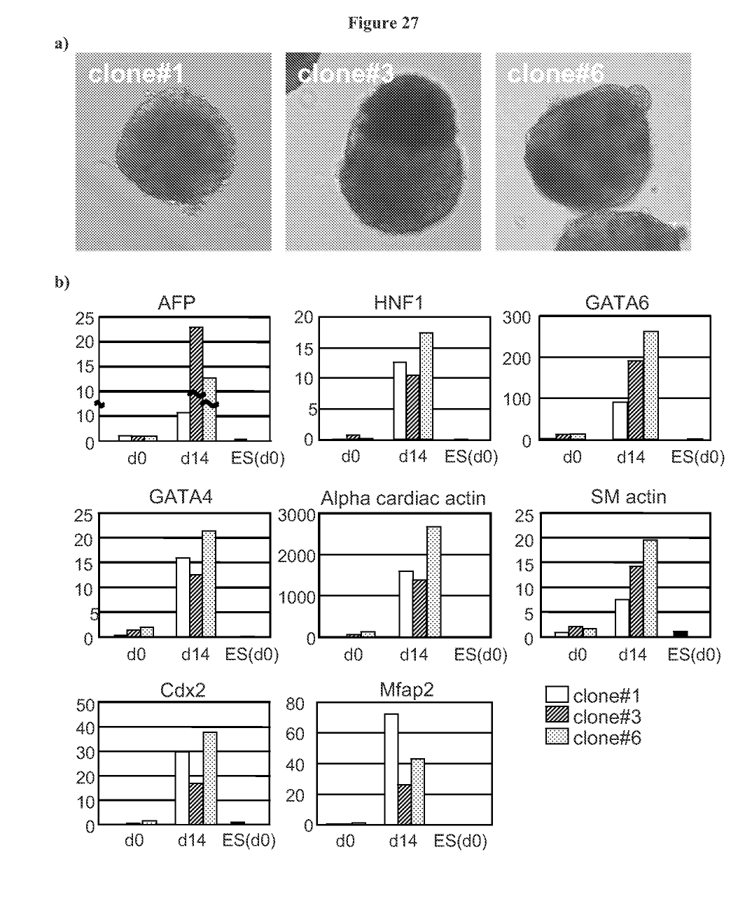

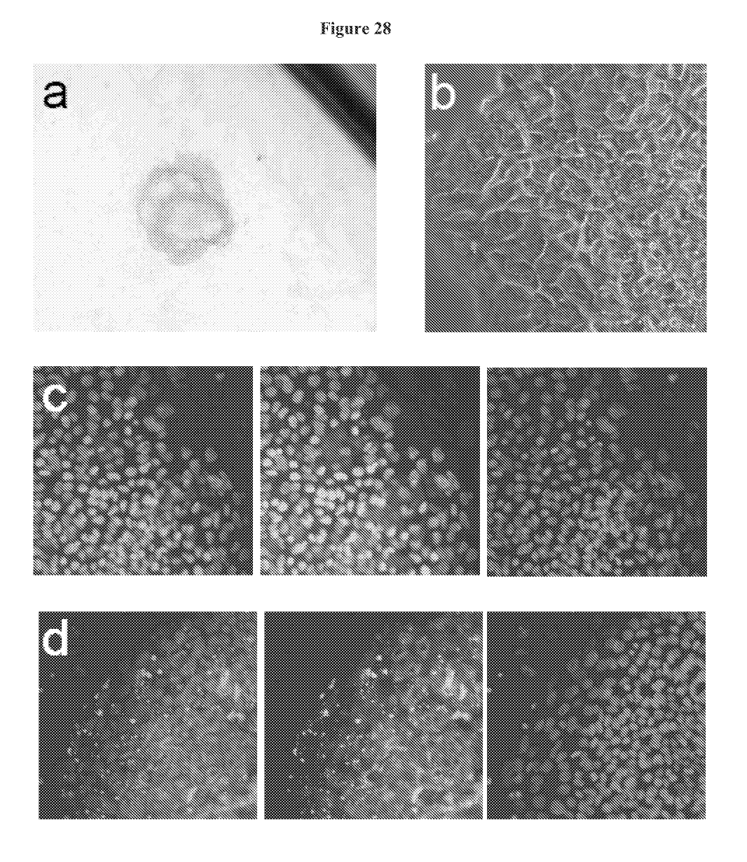

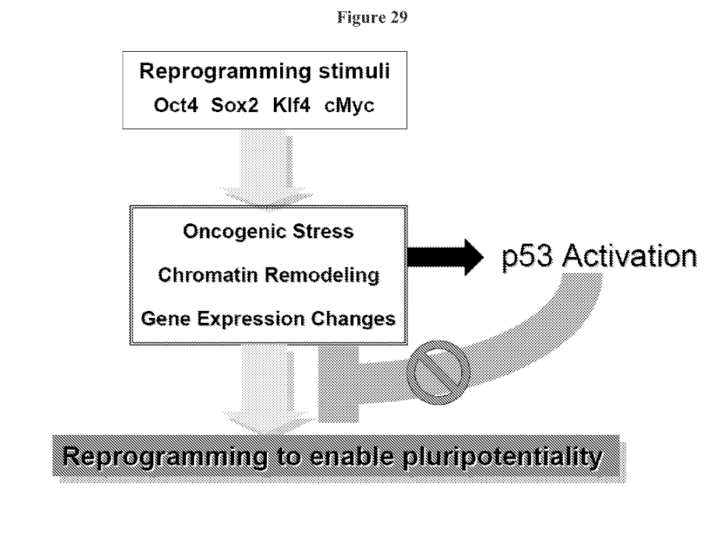

Next it was tested whether downregulating p53 activity had any effect on the reprogramming of human somatic cells. For this purpose, human embryonic fibroblasts (HEFs) and juvenile epidermal keratinocytes were used. Nanog-positive colonies could not be obtained from HEFs with either 3-F or 4-F combined with control shRNA under the present reprogramming conditions after up to 4 weeks. However, ES-like colonies appeared rapidly (after ˜2 weeks) and efficiently from HEFs infected with 3-F or 4-F and p53 shRNA. See FIGS. 5a, 23 and 24, and Table 2. p53 shRNA-induced ES-like colonies exhibited good morphology with regards to expression of human ES marker genes, could be successfully cloned and expanded, and could differentiate in vitro in embryoid body (EB) formation assays. See FIGS. 25-26. Human primary keratinocytes were also more efficiently reprogrammed when p53 activity was experimentally downregulated. In this case, a dominant negative mutant of p53 (p53-DD)²³ with a 288-aa deletion that maintains the initial 14 amino acids and the oligomerization and C-terminal domains was used. p53-DD inhibits p53 activity more effectively than p53 shRNA, as indicated by the inability of Nutlin3a to reduce reprogramming of 3-F or 4-F p53-DD expressing iPS cells (FIG. 5c). Co-transduction of human keratinocytes with 4-F or 3-F and p53-DD resulted in ˜4- and ˜6-fold increase, respectively, in reprogramming efficiency, when compared with co-transduction with a control retrovirus (FIG. 5c), as judged by the number of ES-like colonies that stained positive for AP activity. In contrast, incubation with the p53 agonist Nutlin3a markedly reduced the reprogramming efficiency of human keratinocytes by either 3-F or 4-F (FIG. 5c). iPS-like colonies generated by 3-F or 4-F and p53-DD grew robustly after picking and showed strong expression of pluripotency-associated transcription factors and surface markers (FIG. 5d and data not shown). Furthermore, 3-F-p53-DD iPS cells readily differentiated in vitro into derivatives of the 3 embryonic germ layers, endoderm, ectoderm and mesoderm derivatives as judged by cell morphology and specific immunostaining with α-fetoprotein, Tuj1, and α-actinin, respectively (FIG. 5e). These results show that the reprogramming of human somatic cells to pluripotency, as is the case for mouse cells, is limited by p53 activity.

The present data show that reprogramming somatic cells to iPS cells is associated with activation of the p53 pathway, and this limits the efficiency of reprogramming. Increases in reprogramming efficiency were achieved by reducing or eliminating p53 itself, by interfering with signaling to p53 by reducing the levels of stability modulators such as Arf, or by increasing the effectiveness of a critical negative regulator, Mdmx. The mechanisms by which p53 antagonizes reprogramming appear to involve both its ability to limit cell cycling through induction of the cyclin dependent kinase inhibitor p21 and its ability to induce apoptosis. The similarity of the cell cycle in iPS and ES cells, which both lack significant G1 and G2 periods, imply that p53's ability to restrain the cell cycle at multiple points contribute to its ability to limit reprogramming. Consistent with this, reducing p21 levels, or increasing competence for entering the cell cycle by compromising control of the Rb tumor suppressor through reduction of p16Ink4a levels increased reprogramming. In addition, inhibiting the apoptotic pathway during reprogramming also enhances reprogramming efficiency. This suggests that direct chemical inhibition of the apoptotic cascade may provide a useful tool for enhancing reprogramming efficiency without direct genetic manipulation of tumor suppressors.

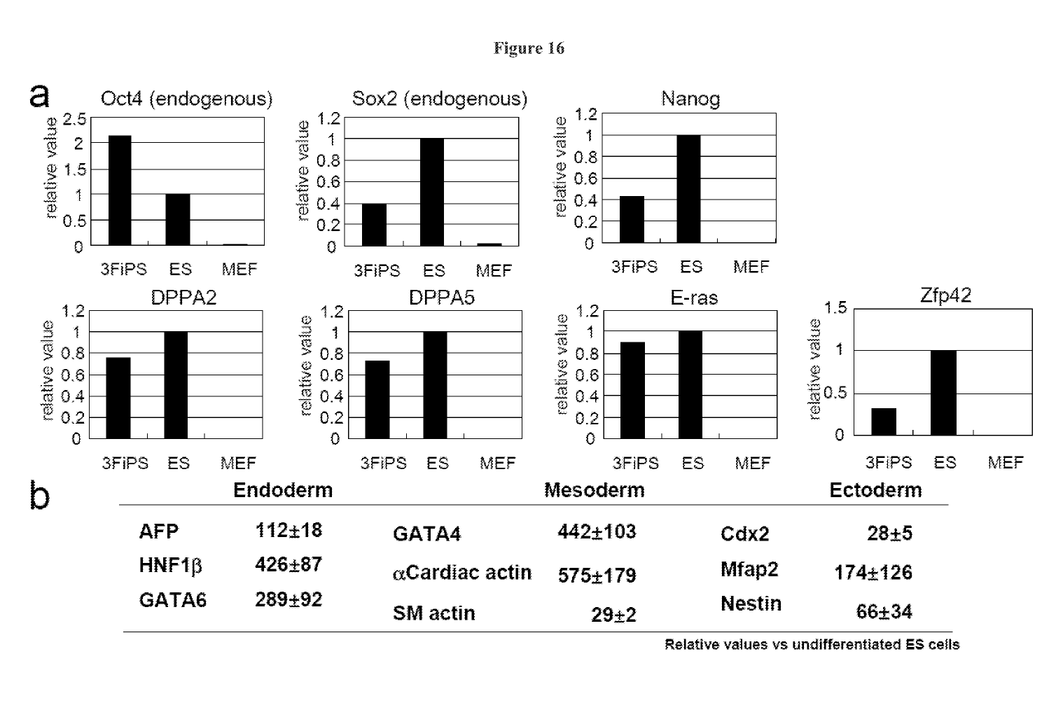

It should be noted that the increased efficiencies reported are likely underestimates of the antagonistic potential of p21, p16Ink4a, and Arf as in each case where shRNA was employed, only partial knock-down of the respective gene products was achieved. The expression of 2-F or 3-F at the levels required for reprogramming also may activate p53 responsive genes that sensitize cells to apoptosis, since overexpression of the anti-apoptosis factor Bcl-2 increased 3-F reprogramming in cells expressing p53. Oct4, Sox2 and Nanog interact with each other to enable the genome-wide chromatin remodeling required for induction of pluripotency. None of these factors are expressed at detectable levels in somatic cells. Previous work showed that p53 represses Nanog in response to DNA damage in ES cells²⁴, raising the possibility that p53 might prevent Nanog expression in MEFs. However, it was observed that Nanog mRNA was not expressed at detectable levels in either p53 wild type or p53-null MEFs. See FIG. 16a. On the other hand, the oncogene Klf4 has been reported to induce Nanog²⁵. It is possible that in the absence of Klf4 in 2-F iPS, p53 elimination allowed Oct4 and Sox2 to remodel the chromatin to a threshold required for expression of sufficient Nanog to drive the subsequent events involved in iPS cell generation.

The present data show that reprogramming in the absence of oncogenes such as c-Myc and Klf4 will require inactivating the p53 and Rb tumor suppressors. While p53 pathway inactivation will be key, this cannot be done on a permanent basis as this would increase the probability of malignant transformation and the generation of unstable genomes that would mitigate use for understanding many diseases. Rather, transient inhibition using chemical antagonists or reversible approaches that avoid genetic disruption will be required²⁶′²⁷. Similarly, as Oct4 and Sox2 exhibit oncogenic characteristics when overexpressed, use of small molecules to transiently mimic their reprogramming functions²⁸⁻³² may enable iPS cells lacking oncogenic alterations to be obtained at acceptable frequencies.

### VI. Materials and Methods

### Reagents

Reagents were obtained from the following sources: Nutlin3a (Cayman Chemical); anti-Oct-3/4 (sc-5279), anti-GKLF (sc-20691), anti-p53 (sc-6243), anti-p21 (sc-53870), anti-p16Ink4a (sc-1207), anti-c-Myc (sc-764) and anti GATA4 (sc-9053) (Santa Cruz Biotechnology); anti-Sox2 (AB5603) (CHEMICON); anti-p53 antibody (1C12), anti-phospho-Histone H2A.X (Ser139) antibody (20E3) (Cell Signaling); anti-Arf (ab80) and anti-Nanog (ab21603) (Abcam); anti-Nanog (SC1000) and anti-p53 (DO-1) (Calbiochem); anti-Tuj1 antibody (MMS-435P-0) (Covance); anti-α-Tubulin (T5168), anti-α-actinin sarcomeric (A7811), anti-α-actin sarcomeric (A2172), anti-Actin (A2066) and anti-chondroitin (C8035) (SIGMA); anti-Foxa2 antibody (AF2400) (R&D systems); anti-alpha-1-fetoprotein (A008) and anti-GFAP (Z0334) (Dako); Anti-TRA-1-81 antibody (Millipore).

### Mice

Wild-type MEFs used for iPS cell production were derived from embryos obtained by mating BDF1/ICR and ICR strains. p53 KO mice were purchased from Taconic Farms, Inc. p53−/− MEFs were obtained by heterozygous versus heterozygous mating. For genotyping, PCR primers are available on the company website. Mdmx mutant mice were generated from ES cells of 129Sv origin by homologous recombination¹⁹.

### Plasmids

Mouse p53 and GFP cDNAs were cloned into pMXs retroviral vectors³³. The cDNA of mouse Bcl-2 was cloned into HIV pBOBI lentiviral vector³⁴. Human p53-DD (a kind gift from Oren, M.) is in pLXSN (Clonetech). The cDNAs of mouse p53 and p21, pMXs-Oct4, -Sox2, -Klf4 and c-Myc were purchased from Addgene^{1,35,36}. Human pMSCV-Oct4, -Sox2, -Klf4 and -c-Myc were constructed as previously described⁶. The short-hairpin RNA (shRNA) sequences against p53, p21, Arf and Ink4a were inserted into pLVTHM lentiviral vectors³⁷. Sequences for shRNA are shown in Table 3.

### Production of Retroviruses and Lentiviruses and iPS Formation

VSV-G viruses were produced in HEK293T cells. For pMX-based and pMSCV-based retroviruses, vectors were transfected using CaPO₄ or lipofectamin, following the manufacturers' directions. One day after transfection, culture medium was changed to new medium. For lentivirus, pBOBI-based³⁴ or pLVTHM-based³⁷ vectors were transfected by Lipofectamine™ 2000 (Invitrogen) according to the manufacturer's protocol. Six hours after transfection, the DNA-lipofectamine-complex was removed and the medium was replaced the next day. Two days after transfection, the supernatant containing viruses was collected and filtered through a 0.45 μm filter. Mouse iPS cells were induced as previously described^{38,39}. Briefly, mouse embryonic fibroblasts (passage 3 to 5) were infected (day 0) with pMX-based retroviruses together with pLVTHM-based lentivirus for shRNAs or pBOBI-based lentivirus for Bcl-2. On day 2, cells were passed onto new gelatin-coated plates. Medium was changed every 2 days. On day 12 to 14, cells were fixed for immunofluorescence study. For the Nutlin3a experiments, cells were treated starting from day 4. Reprogramming of human embryonic fibroblasts (IMR90) was done as previously described²′³. Briefly, IMR90 fibroblasts (passage 7 to 9) were infected (day 0) with pMSCV-based retroviruses+pLVTHM-based lentiviruses for p53 shRNA. On day 4 or 5, cells were passed onto feeder MEFs. Medium was changed every day. Around 3 weeks after infection, cells were fixed for immunofluorescence studies. Reprogramming of human primary keratinocytes was carried out essentially as described⁶. Cells were co-infected with retroviral supernatants containing 3 or 4 reprogramming factors and p53-DD or GFP at a 1:2 ratio. To assess the reprogramming efficiency, cells were trypsinized 3 days after retroviral infection and 10⁴ cells were plated onto 6-cm tissue culture dishes on top of irradiated human foreskin fibroblasts with hES cell medium. After 2 weeks, the dishes were stained for alkaline phosphatase activity and colonies that displayed strong staining and showed hES-like morphology were scored positive.

Protein and mRNA Analysis

Cells were washed once in PBS and lysed in 2×SDS-PAGE sample buffer without 2-mercaptoethanol and glycerol. Lysates were briefly sonicated and cleared by centrifugation. The protein concentration was determined by a BCA Protein Assay Kit (Thermo Scientific). Lysates were then mixed with 2-mercaptoethanol, BPB and glycerol, and boiled. Equal amounts of proteins were subjected to SDS-PAGE. Total RNA was isolated using Trizol® (Invitrogen) followed by cDNA synthesis using Superscript™ II Reverse Transcriptase (Invitrogen). Quantitative PCR was performed using SYBR® GREEN PCR Master Mix (Applied Biosystems).

### Promoter Methylation Analysis

Genomic DNA was isolated and bisulfite modification performed using the EZ DNA Methylation-Direct™ Kit (ZYMO RESEARCH). The promoter regions of Nanog and Oct4 were amplified by nested PCR using primer sets previously described⁴⁰. The amplified PCR products were ligated into pCRII-TOPO (Invitrogen) and sequenced. Data was analyzed using Lasergene (DNASTAR®).

### In Vitro and In Vivo Differentiation

For in vitro differentiation of mouse iPS cells, after dissociation with trypsin/EDTA, cells were cultured in suspension by the hanging drop method. For in vivo differentiation, cells were trypsinized, and injected subcutaneously into SCID mice. After 3 weeks, teratomas were dissected, fixed, and analyzed. Detailed methods for in vitro differentiation, teratoma formation and immunostaining are described herein and/or are known in the art. In vitro differentiation of HEF-derived human iPS cells was induced by culturing cells in suspension and then transferring onto gelatine-coated dish. In vitro differentiation of keratinocytes-derived human iPS cells was carried out as previously described⁶.

### Blastocyst Injections for Chimeric Mice

iPS cells were injected into C57BL/6J hosts blastocysts and transferred into 2.5 dpc ICR pseudo-pregnant recipient females. Chimerism was ascertained after birth by the appearance of agouti coat color (from iPS cell) in black host pups.

### VII. Tables

A summary of reprogramming efficiency in mouse fibroblasts is tabulated in Table 1 following. The number of Nanog-positive colonies was calculated after immunostaining at d12-14. The total number of Nanog-positive colonies was divided by the number of infected cells. Error numbers indicate s.d. The term “s.d.” refers in the customary sense to standard deviation. The term “s.e.” refers in the customary sense to standard error. Student's t-tests were performed for statistical analysis. p=0.007 (p53-shRNA vs mock, n=4 in each), p=0.0006 (p53−/− vs WT, n=3 in each), p=0.009 (p21-shRNA vs mock, n=4 in each), p=0.0005 (Arf-shRNA vs mock, n=3 in each), p=0.028 (Arf/Ink4a-shRNA vs mock, n=3 in each), p=0.010 (Ctl(solvent) vs Nutlin-3(10 μM), n=3 in each), p=0.045 (3SA/3SA vs WT, n=3 in each), p=0.022 (Bcl-2 vs mock, n=4 in each), and p=0.012 (c-Myc versus mock, n=4 in each).

TABLE 1
Reprogramming efficiency in mouse fibroblasts

| | | | |
|---|---|---|---|
| 3F⁺ | ↑↑ | p53 shRNA | 0.421 + 0.170% vs 0.114 + 0.054% |
| | ↑↑↑ | p53 KO | 2.278 + 0.438% vs 0.197 + 0.060% |
| | ↑_{↑↑} | p21 shRNA | 0.359 + 0.144% vs 0.114 + 0.054% |
| | ↑ | Arf shRNA | 0.163 + 0.013% vs 0.085 + 0.083% |
| | ↑↑ | Arf/Ink4a shRNA | 0.366 + 0.144% vs 0.114 + 0.054% |
| | ↓↓ | Mdm2 inhibition | 0.007 + 0.008% vs 0.114 + 0.054% |
| | ↑↑ | Mdmx mutant | 0.259 + 0.167% vs 0.041 + 0.028% |
| | | (3SA) | |
| | ↑↑ | Bcl-2 expression | 0.268 + 0.117% vs 0.100 + 0.062% |
| | ↑↑ | c-Myc (4 factors) | 0.517 + 0.264% vs 0.114 + 0.054% |
| 2F⁺ | ↑ | p53 shRNA | 0.003 + 0.002% vs 0.000% |

A summary of reprogramming efficiency of human embryonic fibroblasts (HEFs) is tabulated in Table 2 following. The number of Nanog- or Tral-81-positive colonies was calculated after immunostaining at dl 8-27. No colonies were observed from mock +4-F or 3-F cells in all trials. The total number of colonies was divided by the number of infected cells. Error numbers indicate s.d (n=4).

TABLE 2
Reprogramming efficiency of HEFs.

| | 3-F | | 4-F | |
|---|---|---|---|---|
| Efficiency | mock | p53shRNA | mock | p53shRNA |
| | 0% | 0.027% * | 0% | 0.153 ± 0.188% |

| | |
|---|---|
| | $( \begin{matrix}0.208 \pm 0.240 \% \\ by Tra 1 \text{-} 81\end{matrix} )$ |
| 0 colonies/2 × 10₄ cells (mock) | |

Actual number of Nanog-positive

| 3-F | | 4-F | |
|---|---|---|---|
| Trial 1 | 0 colonies/3 × 10⁴ cells (mock) | Trial 1 | 0 colonies/1 × 10⁴ cells (mock) |
| | 10 colonies/3 × 10⁴ cells (p53shRNA) | | 86 colonies/2 × 10⁴ cells (p53shRNA) |
| Trial 2 | 0 colonies/2 × 10⁴ cells (mock) | Trial 2 | 0 colonies/3 × 10⁴ cells (mock) |
| | 2 colonies/1 × 10⁴ cells (p53shRNA) | | 13 colonies/2 × 10⁴ cells (p53shRNA) |
| | | Trial 3 | 0 colonies/2 × 10⁴ cells (mock) |
| | | | 19 colonies/2 × 10⁴ cells (p53shRNA) |
| | | Trial 4 | 0 colonies/2 × 10⁴ cells (mock) |
| | | | 4 colonies/2 × 10⁴ cells (p53shRNA) |

* Average of 2 independent experiments

Sequences of shRNA useful in the methods described herein are tabulated in Table 3 following.

TABLE 3
shRNA sequences

| | | SEQ ID | |
|---|---|---|---|
| Gene | Sequence | NO: | Reference^{a} |
| GFP(control) | GAAGCAGCACGACTTCTTC | 1 | 1 |
| mouse p53#1 | GACTCCAGTGGGAACCTTC | 2 | 2 |
| mouse p53#2 | GTACATGTGTAATAGCTCC | 3 | 3 |
| p21 | TTAGGACTCAACCGTAATA | 4 | 4 |
| Arf | CACCGGAATCCTGGACCAG | 5 | 5 |
| Arf/Ink4a | AATGGCTGGATTGTTTAAA | 6 | 4 |
| human p53#1 | GACTCCAGTGGTAATCTAC | 7 | 2 |

^{a}References:
(1): Takehara A, et al., 2007, Cancer Res. 67, 9704-12;
(2): Brummelkamp, T. R., et al., 2002, Science 296, 550-553;
(3): Tiscornia, G., et al., 2004, Proc. Natl. Acad. Sci. U.S.A. 101, 7347-7351;
(4): Fasano, C. A., et al., 2007, Cell Stem Cell 1, 87-99;
(5): Sage, J., et al., 2003, Nature 424, 223-228.

### VIII. Bibliography

Following are the references cited to herein: (1): Takahashi, K. and Yamanaka, S, 2006, Cell 126, 663-76; (2): Takahashi, K. et al., 2007, Cell 131, 861-72; (3): Yu, J. et al., 2007, Science 318, 1917-20; (4): Park, I. H. et al., 2008, Nature 451, 141-6; (5): Lowry, W. E. et al., 2008, Proc Natl Acad Sci USA. 105, 2883-8; (6): Aasen, T. et al., 2008, Nat. Biotechnol. 26, 1276-84; (7): Nakagawa, M. et al., 2008, Nat. Biotechnol. 26, 101-6; (8): Wernig, M., Meissner, A., Cassady, J. P. and Jaenisch, R., 2008, Cell Stem Cell 2, 10⁻²; (9): Okita, K., Ichisaka, T. and Yamanaka, S., 2007, Nature 448, 313-7; (10): Rowland, B. D., Bernards, R. and Peeper, D. S., 2005, Nat Cell Biol. 7, 1074-82; (11): Kanatsu-Shinohara, M. et al., 2004, Cell 119, 1001-12; (12): Cleveland, J. L. and Sherr, C. J., 2004, Cancer Cell 6, 309-311; (13): Vassilev, L. T. et al., 2004, Science 303, 844-8; (14): Miyashita, T. and Reed, J. C., 1995, Cell 80, 293-9; (15): Kamijo, T. et al., 1998, Proc. Natl. Acad. Sci. USA. 95, 8292-8297; (16): Pomerantz, J. et al., 1998, Cell 92, 713-723; (17): Zhang, Y., Xiong, Y. and Yarbrough, W. G., 1998, Cell 92, 725-734; (18): Knudsen, E. S, and Knudsen, K. E., 2008, Nature Rev. Cancer 8, 714-24; (19): Wang, V. Y., LeBlanc, M., Wade, M., Jochemsen, A. G. and Wahl, G. M, Manuscript submitted; (20): Marine, J. C., Dyer, M. A. and Jochemsen, A. G., 2007. J Cell Sci. 120, 371-8; (21): Berkovich, E., Monnat, R. J. Jr. and Kastan, M. B., 2007, Nat Cell Biol. 9, 683-90; (22): Foster, K. W. et al., 1999, Cell Growth Differ. 10, 423-34; (23): Shaulian, E., Zauberman, A., Ginsberg, D., and Oren, M., 1992, Mol Cell Biol. 12, 5581-92; (24): Lin, T. et al., 2005, Nat Cell Biol. 2, 165-71; (25): Jiang, J. et al, 2008. Nat Cell Biol. 10, 353-60; (26): Komarov, P. G. et al., 1999, Science 285, 1651-53; (27): Zhao, Y. et al., 2008, Cell Stem Cell 3, 475-9; (28): Shi, Y. et al., 2008, Cell Stem Cell 6, 568-74; (29): Huangfu, D. et al., 2008, Nat. Biotechnol. 26, 1269-75; (30): Woltjen, K. et al., 2009, Nature in advance online publication; (31): Kaji, K. et al., 2009, Nature in advance online publication; (32): Soldner, F. et al., 2009, Cell 136, 964-77; (33): Kitamura, T. et al., 2003, Exp Hematol. 31, 1007-14; (34): Miyoshi, H., Blömer, U., Takahashi, M., Gage, F. H. and Verma, I. M., 1998, J. Virol. 72, 8150-7; (35): Sherley, J. L., 1991, J Biol. Chem. 266, 24815-28; (36): Huppi, K. et al., 1994, Oncogene 9, 3017-20; (37): Wiznerowicz, M. and Trono, D., 2003, J. Virol. 77, 8957-61; (38): Blelloch, R., Venere, M., Yen, J. and Ramalho-Santos, M., 2007, Cell Stem Cell 1, 245-247; (39): Takahashi, K., Okita, K., Nakagawa, M. and Yamanaka, S., 2008, Nat. Protoc. 2, 3081-9; and(40): Blelloch, R. et al., 2006, Stem Cells 24, 2007-13.

1. A method for preparing an induced pluripotent stem cell comprising:
(i) transfecting a non-pluripotent cell with a nucleic acid encoding an Oct4 protein, a nucleic acid encoding a Sox2 protein and a nucleic acid encoding a p53 inhibitor to form a transfected non-pluripotent cell; and
(ii) allowing said transfected non-pluripotent cell to divide thereby forming said induced pluripotent stem cell.

2. (canceled)

3. (canceled)

4. The method of claim 1, wherein said nucleic acid encoding an Oct4 protein and said nucleic acid encoding a Sox2 protein form part of a first nucleic acid and said nucleic acid encoding a p53 inhibitor form part of a second nucleic acid.

5. The method of claim 1, wherein said p53 inhibitor is a p53-specific short hairpin RNA.

6. The method of claim 1, wherein said p53 inhibitor is a dominant negative p53 protein.

7. A method for preparing an induced pluripotent stem cell comprising:
(i) transfecting a p53-deficient non-pluripotent cell with a nucleic acid encoding an Oct4 protein and a nucleic acid encoding a Sox2 protein to form a transfected p53-deficient non-pluripotent cell; and
(ii) allowing said transfected p53-deficient non-pluripotent cell to divide thereby forming said induced pluripotent stem cell.

8. A method for preparing an induced pluripotent stem cell comprising:
(i) introducing a p53 inhibitor to a non-pluripotent cell;
(ii) transfecting said non-pluripotent cell with a nucleic acid encoding an Oct4 protein and a nucleic acid encoding a Sox2 protein to form a transfected non-pluripotent cell; wherein transfecting said non-pluripotent cell is performed before, after or at the same time of introducing said p53 inhibitor to said non-pluripotent cell; and
(iii) allowing said transfected non-pluripotent cell to divide thereby forming said induced pluripotent stem cell.

9. The method of claim 8, wherein said p53 inhibitor is a chemical compound.

10. The method of claim 8, wherein said p53 inhibitor is a small molecule.

11. (canceled)

12. (canceled)

13. (canceled)

14. (canceled)

15. (canceled)

16. A non-pluripotent cell comprising a nucleic acid encoding an Oct4 protein, a nucleic acid encoding a Sox2 protein and a nucleic acid encoding a p53 inhibitor.

17. The non-pluripotent cell of claim 16, wherein said non-pluripotent cell consists essentially of a nucleic acid encoding an Oct4 protein, a nucleic acid encoding a Sox2 protein and a nucleic acid encoding a p53 inhibitor.

18. (canceled)

19. (canceled)

20. The non-pluripotent cell of claim 16, wherein said nucleic acid encoding an Oct4 protein and said nucleic acid encoding a Sox2 protein form part of a first nucleic acid and said nucleic acid encoding a p53 inhibitor form part of a second nucleic acid.

21. The non-pluripotent cell of claim 16, wherein said non-pluripotent cell is a human cell.

22. (canceled)

23. The non-pluripotent cell of claim 16, wherein said p53 inhibitor is a p53-specific short hairpin RNA.

24. The non-pluripotent cell of claim 16, wherein said p53 inhibitor is a dominant negative p53 protein.

25. A p53-deficient non-pluripotent cell comprising a nucleic acid encoding an Oct4 protein and a nucleic acid encoding a Sox2 protein.

26. The p53-deficient non-pluripotent cell of claim 25, wherein said p53-deficient non-pluripotent cell consists essentially of a nucleic acid encoding an Oct4 protein and a nucleic acid encoding a Sox2 protein.

27. A non-pluripotent cell comprising a nucleic acid encoding an Oct4 protein, a nucleic acid encoding a Sox2 protein and a p53 inhibitor.

28. The non-pluripotent cell of claim 27, wherein said non-pluripotent cell consists essentially of a nucleic acid encoding an Oct4 protein, a nucleic acid encoding a Sox2 protein and a p53 inhibitor.

29. A method of treating a mammal in need of tissue repair comprising:
(a) administering an induced pluripotent stem cell to said mammal;
(b) allowing said induced pluripotent stem cell to divide and differentiate into somatic cells in said mammal, thereby providing tissue repair in said mammal;
wherein said induced pluripotent stem cell is prepared by a process comprising the steps of:
(i) transfecting a non-pluripotent cell with a nucleic acid encoding an Oct4 protein, a nucleic acid encoding a Sox2 protein and a nucleic acid encoding a p53 inhibitor to form a transfected non-pluripotent cell; and
(ii) allowing said transfected non-pluripotent cell to divide thereby forming said induced pluripotent stem cell;
or wherein said pluripotent stem cell is prepared by a process comprising the steps of:
(i) transfecting a p53-deficient non-pluripotent cell with a nucleic acid encoding an Oct4 protein, a nucleic acid encoding a Sox2 protein to form a transfected non-pluripotent cell; and
(ii) allowing said transfected non-pluripotent cell to divide thereby forming said induced pluripotent stem cell;
or wherein said induced pluripotent stem cell is prepared by a process comprising the steps of:
(i) introducing a p53 inhibitor to a non-pluripotent cell;
(ii) transfecting said non-pluripotent cell with a nucleic acid encoding an Oct4 protein and a nucleic acid encoding a Sox2 protein to form a transfected non-pluripotent cell; wherein transfecting said non-pluripotent cell is performed before, after or at the same time of introducing said p53 inhibitor to said non-pluripotent cell; and
(iii) allowing said transfected non-pluripotent cell to divide thereby forming said induced pluripotent stem cell.

30. A method for producing a somatic cell comprising:
(a) contacting an induced pluripotent stem cell with a cellular growth factor; and
(b) allowing said induced pluripotent stem cell to divide, thereby forming said somatic cell;
wherein said induced pluripotent stem cell is prepared by a process comprising the steps of:
(i) transfecting a non-pluripotent cell with a nucleic acid encoding an Oct4 protein, a nucleic acid encoding a Sox2 protein and a nucleic acid encoding a p53 inhibitor to form a transfected non-pluripotent cell; and
(ii) allowing said transfected non-pluripotent cell to divide thereby forming said induced pluripotent stem cell;
or wherein said induced pluripotent stem cell is prepared by a process comprising the steps of:
(i) transfecting a p53-deficient non-pluripotent cell with a nucleic acid encoding an Oct4 protein, a nucleic acid encoding a Sox2 protein to form a transfected non-pluripotent cell; and
(ii) allowing said transfected non-pluripotent cell to divide thereby forming said induced pluripotent stem cell;
or wherein said induced pluripotent stem cell is prepared by a process comprising the steps of:
(i) introducing a p53 inhibitor to a non-pluripotent cell;
(ii) transfecting said non-pluripotent cell with a nucleic acid encoding an Oct4 protein and a nucleic acid encoding a Sox2 protein to form a transfected non-pluripotent cell; wherein transfecting said non-pluripotent cell is performed before, after or at the same time as said introducing said p53 inhibitor to said non-pluripotent cell; and
(iii) allowing said transfected non-pluripotent cell to divide thereby forming said induced pluripotent stem cell.
